(19) United States
(12) Patent Application Publication
(10) Pub. No.: US 2015/0374673 A1
(43) Pub. Date: Dec. 31, 2015

(54) Combination Therapy for the Treatment of Nosocomial Pneumonia

(71) Applicant: ASTRAZENECA AB, Södertälje (SE)

(72) Inventors: Shampa Das, Cheshire (GB); Jianguo Li, Waltham, MA (US); Johan Willem Mouton, Nijmegen (NL); Wright Nichols, Waltham, MA (US)

(73) Assignee: AstraZeneca AB, Sodertalje (SE)

(21) Appl. No.: 14/765,606

(22) PCT Filed: Feb. 6, 2014
(86) PCT No.: PCT/GB2014/050354

§ 371 Date: Aug. 4, 2015

Related U.S. Application Data
(60) Provisional application No. 61/761,369, filed on Feb. 6, 2013.

(51) Int. Cl. A61K 31/439; A61K 38/14; A61K 31/5383; A61K 31/65; A61K 9/00; A61K 31/5377; A61K 31/546; A61K 31/7036

(52) U.S. Cl. CPC A61K 31/439; A61K 31/546; A61K 38/14; A61K 31/7036; A61K 31/65; A61K 9/0019; A61K 31/5377; A61K 31/5383

(57) ABSTRACT

The present invention relates to a method of treatment of nosocomial pneumonia using a combination of ceftazidime (a third generation cephalosporin) and avibactam (a novel β-lactamase inhibitor), optionally with one or more additional therapeutic agents.

### FIELD OF THE INVENTION

The present invention relates to a method of treatment of nosocomial pneumonia using a combination of ceftazidime (a third generation cephalosporin) and avibactam (a novel β-lactamase inhibitor), optionally with one or more additional therapeutic agents.

### BACKGROUND OF THE INVENTION

The international microbiological and infectious disease community continues to express serious concern that the continuing evolution of antibacterial resistance could result in bacterial strains against which currently available antibacterial agents will be ineffective. The outcome of such an occurrence could have considerable morbidity and mortality.

In the fight against bacterial infection, beta-lactam antibiotics are essential. Beta-lactams are a broad class of drugs which all have a beta-lactam in their core molecular structure, and typically show effectiveness against a broad spectrum of Gram-positive and Gram-negative bacteria by inhibiting the cell wall synthesis of the bacterium. Because the drug target has no eukaryotic analog, their toxicity is low and they are generally well-tolerated. Beta-lactam antibiotics include penicillin derivatives (penams), cephalosporins, monobactams and carbapenems. They remain among the most widely prescribed, safe and effective drugs available to combat bacterial infection. However, their effectiveness is limited by highly resistant infectious strains such as methicillin-resistant Staphylococcus aureus (MRSA) and multi-drug resistant (MDR) strains of Pseudomonas aeruginosa, Acinetobacter baumannii, Escherichia coli, Klebsiella pneumonia, and other Enterobacteriaceae. Such resistant bacteria are major causes of patient morbidity and mortality. Helfand, β-lactams Against Emerging ‘Superbugs’: Progress and Pitfalls, Expert Rev. Clin. Pharmacol. 1(4):559-571 (2008).

To help improve the effectiveness of beta-lactam antibiotics, some beta-lactamase inhibitors have been developed. However, the currently available β-lactamase inhibitors in many instances are insufficient to counter the constantly increasing diversity of β-lactamases. The three most common serine beta-lactamase agents currently used clavulanic acid, tazobactam and sulbactam have activity only against certain Class A enzymes, which severely limits their utility. Newer beta-lactamase inhibitors currently in clinical trials, such as Avibactam, work both on Class A and C enzymes, with some limited effectiveness against Class D beta-lactamases. Bebrone, et al., Current Challenges in Antimicrobial Chemotherapy: Focus on β-Lactamase Inhibition, Drugs, 70(6):651- [symbol] 679 (2010).

Beta-lactam antibiotics, alone and in combination with beta-lactamase inhibitors, continue to represent an essential portion of the antibacterial agents used to combat disease. β-lactam resistance for Gram-negative infections is primarily driven by β-lactamase activity; and the significant dependence on β-lactam antibiotics has lead to the diversification and increased prevalence of β-lactamases. These β-lactamases are driving resistance to even the newest β-lactam antibiotics. Llarrull, et al., The Future of Beta-Lactams, Current Opinion in Microbiology, 13:551-557 (2010). Extended-spectrum β-lactamase (ESBL)-, AmpC-, KPC-, NDM- and OXA-48-producing Enterobacteriaceae as well as Acinetobacter baumannii and Pseudomonas aeruginosa are amongst the most important and frequently isolated nosocomial pathogens and are often resistant to many classes of antibiotics. D. M. Livermore, et al. Activities of NXL104 Combinations with Ceftazidime and Aztreonam Against Carbapenemase-Producing Enterobacteriacea, Antimicrobial Agents Chemotherapy, 55 (2011), pp. 390-394; S. Mushtaq, et al., In Vitro Activity of Ceftazidime+NXL104 Against Pseudomonas aeruginosa and other Non-Fermenter; J Antimicrobial Chemotherapy, 65 (2010) 2376-381; A. Endimiani, et al., In Vitro Activity of NXL104 in Combination with β-Lactams Against Klebsiella pneumonia Isolates Producing KPC Carbapenemases; Antimicrobial Agents Chemotherapy, 53 (2009) 3599-3601.

Nosocomial pneumonia refers to any pneumonia contracted by a patient in a hospital at least 48-72 hours after being admitted and includes hospital-acquired pneumonia (HAP) and ventilator-associated pneumonia (VAP). Of patients with nosocomial pneumonia infections, HAP accounts for about 70% of nosocomial pneumonia patients, and the remaining approximately 30% have VAP. In patients with HAP, hospital mortality rates range from 12-35% (Freire et al 2010; Chung et al 2011) but actual rates are often associated with the patients underlying condition. Patients with VAP are recognized as being a more seriously ill population with attributable mortality rates cited in the range 33-50% (Am J Respir Crit Care Med, 2005, 171, pp 388).

There is a significant need in nosocomial pneumonia for first treatment (empiric) options that are more effective in treating the pathogens most commonly found in nosocomial pneumonia. Confirmation of pathogens takes up to 48 hours, and in some clinical settings such as HAP, detection rates are relatively low (circa 60%) meaning treatment choice is made on suspicion of pathogen and/or the possibility of resistance. There is a significant need for empiric therapy options that are more effective in treating the pathogens most commonly found in nosocomial pneumonia, as existing treatment options for Gram negative pathogens have levels of susceptibility for key resistant pathogens of below 80%. There are few effective empiric agents for treating serious Gram [symbol] negative infection (Pseudomonas aeruginosa, and resistant pathogens expressing Extended Spectrum β-Lactamases (ESBLs), or Klebsiella pneumoniae Carbapenemases (KPCs). The diminishing effectiveness of existing empiric therapeutic options (e.g. carbapenems, cephalosporins) creates a significant need for agents like the ceftazidime-avibactam (CAZ-AVI) combination, which compared to established treatments is active versus a broader spectrum of hard to treat and resistant pathogens. New treatments such as CAZ-AVI are needed for empiric use to increase treatment success rates in patients at higher risk of mortality and morbidity if the empiric therapy is inadequate.

It has been surprisingly and unexpectedly found that the CAZ-AVI combination provides an excellent treatment option for nosocomial pneumonia patients. While the in vitro spectrum of this combination showed promise for treating the major bacterial strains responsible for causing nosocomial pneumonia, our findings show that the combination can penetrate into the target tissues in sufficient quantities to effectively treat the infection.

### SUMMARY OF THE INVENTION

The present invention is directed to use of a combination of ceftazidime and Avibactam to treat nosocomial pneumonia, including HAP and VAP, optionally in combination with one or more additional therapeutic agents. The present invention is also directed to a method of treatment of a nosocomial pneumonia infection in a patient in need thereof comprising administering to the patient an effective amount of the combination of ceftazidime or a pharmaceutically acceptable salt thereof, and Avibactam, or a pharmaceutically acceptable salt thereof. In one embodiment, this combination further comprises administering the combination with one or more an additional therapeutic agents.

### BRIEF DESCRIPTION OF THE DRAWINGS

FIG. 1. Human simulated serum concentration-time profile for ceftazidime-avibactam 2000-500 mg every 8 h as a 2 h infusion in man as compared with serum exposures observed in infected and uninfected female ICR mice. The black line is the human ceftazidime exposure, black circles are ceftazidime serum concentrations of infected mice, black squares are ceftazidime serum concentrations of uninfected mice, the dotted line is the human avibactam exposure, white circles are the avibactam serum concentrations of infected mice, and the white squares are the avibactam serum concentrations of uninfected mice.

FIG. 2. Epithelial lining fluid (ELF) concentration-time profile after human simulated serum doses of ceftazidime-avibactam 2000-500 mg every 8 h as a 2 h infusion in man observed in infected [symbol] and uninfected mice. In A), the black circles are the ELF ceftazidime concentrations in infected, female ICR mice; the black squares are the ELF ceftazidime concentrations in uninfected mice; in B), the black triangles are the ELF avibactam concentrations in infected mice, and the black diamonds are the ELF avibactam concentrations in uninfected mice.

FIG. 3. Serum concentration-time profile after human simulated serum doses of ceftazidime-avibactam 2000-500 mg every 8 h as a 2 h infusion in man as compared with that observed in infected, female, ICR mice. The black line is the human ceftazidime exposure, black circles are ceftazidime serum concentrations in mice, the dotted line is the human avibactam exposure, and white triangles are the avibactam serum concentrations in mice.

FIG. 4. Epithelial lining fluid (ELF) concentration-time profile after human simulated serum doses of ceftazidime-avibactam 2000-500 mg every 8 h as a 2 h infusion in man observed in infected, female ICR mice. The black circles are the ceftazidime ELF concentrations in mice and the black squares are the avibactam ELF concentrations in mice.

FIG. 5. Efficacy of human simulated serum doses of ceftazidime-avibactam 2000-500 mg every 8 hours as a 2 h infusion and associated ELF fT>MIC against P. aeruginosa in the neutropenic lung infection model. (MICs of CAZ-AVI are shown in brackets by each strain name). Bars represent mean±SD

FIG. 6. Serum concentration-time profile after human simulated serum doses of ceftazidime 2000 mg every 8 h as a 2 h infusion in man observed in infected, female, ICR mice. The black circles are ceftazidime serum concentrations in mice, the black squares are the ceftazidime ELF concentrations in mice.

FIG. 7. Efficacy of human simulated serum doses of ceftazidime 2000 mg every 8 hours as a 2 h infusion against P. aeruginosa in the neutropenic lung infection model. (MICs of CAZ are shown in brackets by each strain name). Bars represent mean±SD

FIG. 8. Serum concentration-time profile after a regimen of ceftazidime to produce directed ELF fT>MIC observed in infected, female, ICR mice. The black circles are ceftazidime serum concentrations in mice, the black squares are the ceftazidime ELF concentrations in mice.

FIG. 9. Efficacy of human simulated serum doses of ceftazidime directed ELF fT>MIC and associated ELF fT>MIC against P. aeruginosa in the neutropenic lung infection model. (MICs of CAZ are shown in brackets by each strain name). Bars represent mean±SD

[symbol]

FIG. 10: Exposure response of avibactam in thigh infected mice treated with ceftazidime q2h: dose fractionation

FIG. 11: Exposure response of avibactam in thigh infected mice treated with ceftazidime q2h for 6 P. aeruginosa strains.

FIG. 12: Treatment of lung infected mice with ceftazidime dosing every 2 hours and avibactam every 2 or 8 hours

FIG. 13: Exposure response of avibactam in lung infected mice treated with ceftazidime q2h: dose fractionation

FIG. 14: Exposure response of avibactam in lung infected mice treated with ceftazidime q2h for 4 P. aeruginosa strains

### DETAILED DESCRIPTION OF THE INVENTION

The CAZ-AVI combination demonstrates significant activity against clinically important Gram-negative pathogens (e.g., P. aeruginosa and Enterobacteriaceae, including K. pneumoniae, and Enterobacter species), including those resistant to extended spectrum cephalosporins, piperacillin/tazobactam and carbapenems through ESBL, KPCs, AmpC or OXA-48 β-lactamase production. CAZ-AVI also demonstrates higher rates of susceptibility versus standard of care antibiotics against key local Gram-negative pathogens (eg, P. aeruginosa, and Enterobacteriaceae, including K. pneumoniae) including strains resistant to commonly used antibiotics, including multi-drug resistant strains.

This potent spectrum could provide potential effective coverage for the vast majority of patients with nosocomial pneumonia infections, but only if the drugs can actually penetrate to the site of infection at clinically effective levels. Numerous agents with potentially relevant pathogen efficacy are not able to effectively treat nosocomial pneumonia infections based on their inability to get to the site of infection (penetrate the epithelial lining fluid (ELF)) in effective amounts. Often times, drug load must be significantly increased to provide an effective amount of drug to the site of infection, which increases the potential side-effects suffered by the patient, which in turn can lead to non-compliance with administration schedule or discontinuation of the treatment. Not only does a potential treatment for nosocomial pneumonia require effective ELF penetration, but an effective agent also needs to retain its antibacterial activity in the presence of lung surfactant, and suffer no detrimental drug-drug interactions with additional therapeutic agents which may be co-administered to the patient in an overall treatment regimen. Any one of these considerable hurdles [symbol] can make a potentially attractive antibacterial agent unavailable for the treatment of nosocomial pneumonia infections such as HAP and VAP.

We have surprisingly and unexpectedly found that the CAZ-AVI combination has the required profile to successfully treat nosocomial infections not only does it provide an attractive profile against the major pathogens which cause nosocomial pneumonia infections, but it can effectively penetrate the ELF to reach the site of infection, does not lose efficacy in the presence of lung surfactants, and can be successfully administered with many common agents for a total treatment plan for this extremely ill patient population. The level of avibactam penetration to human ELF (approximately 30%) and the fact that lung surfactant does not impact the efficacy of avibactam to restore the activity of ceftazidime at the site of a nosocomial pneumonia infections, even at the same dose levels used to treat infections at other sites in the body, is surprising and represents a huge advance in the possible treatment options for NP patients.

In one aspect of the invention is a method of treating nosocomial pneumonia in a patient in need there of comprising administering to the patient and effective amount of ceftazidime, or a pharmaceutically acceptable salt thereof, and avibactam, or a pharmaceutically acceptable salt thereof.

Ceftazidime is (6R,7R)-7-[[(2Z)-2-(2-amino-1,3-thiazol-4-yl)-2-(1-hydroxy-2-methyl-1-oxopropan-2-yl)oxyiminoacetyl]amino]-8-oxo-3-(pyridin-1-ium-1-ylmethyl)-5-thia-1-azabicyclo[4.2.0]oct-2-ene-2-carboxylate pentahydrate. The chemical structure is depicted below:

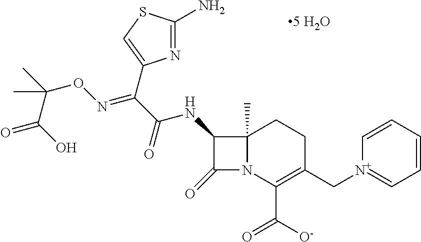 [SMILES: CC(C)(O/N=C(/C(=O)N[C@@H]1C(=O)N2C(C(=O)[O-])=C(C[n+]3ccccc3)CC[C@]12C)c1csc(N)n1)C(=O)O]

Avibactam is [(2S,5R)-2-carbamoyl-7-oxo-1,6-diazabicyclo[3.2.1]octan-6-yl]hydrogen sulfate. The chemical structure is depicted below:

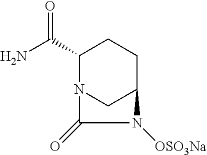

According to some embodiments, the present invention provides methods for treating nosocomial pneumonia infections in patients in need thereof by providing a dosage form comprising about 2000 mg of ceftazidime and about 500 mg of avibactam. In this embodiment, administration of a dosage form constitutes administering a dose of the combination. In one aspect of this embodiment, the patient receives a dose of the combination every 8 hours. In one aspect of this embodiment, the patient receives each dose of the combination via an intravenous infusion. In one aspect of this embodiment, the patient receives each dose of the combination is via an intravenous infusion which is administered over the course of approximately two hours. In one aspect of this embodiment, the patient receives each dose of the combination is via an intravenous infusion which is administered over the course of approximately one hour. In one aspect of this embodiment, the patient receives the combination in a single infusion. In one aspect of this embodiment, the patient receives the combination in a series of infusions.

In some embodiments, the present invention provides compositions consisting essentially of the combination of ceftazidime and avibactam or a pharmaceutically acceptable salt of either or both components thereof. In such compositions, ceftazidime and avibactam are the only active ingredients. An active ingredient as defined herein is one which is effective for the treatment of nosocomial pneumonia infections. Such compositions can have other ingredients that are inactive and/or not antibacterial agents, antimicrobial agents. Examples of such ingredients include, but are not limited to, one or more pharmaceutically acceptable carriers, excipients, additives, or other ingredients useful in formulating the compositions.

One embodiment of the inventions is the combination of ceftazidime, or a pharmaceutically acceptable salt thereof, and avibactam, or a pharmaceutically acceptable salt thereof, for use as a medicament.

One embodiment of the invention is the combination of ceftazidime, or a pharmaceutically acceptable salt thereof, and avibactam, or a pharmaceutically acceptable salt thereof, for use in the treatment of a nosocomial pneumonia infection. In one aspect of this embodiment, the combination is for use in the treatment of a nosocomial pneumonia infection which is caused by one or more [symbol] pathogens which express one or more beta-lactamase. In one aspect of this embodiment, the combination is used for the treatment of a nosocomial pneumonia infection which is not susceptible to ceftazidime as a mono-therapy. In one aspect of this invention, the combination is used for the treatment of a nosocomial pneumonia infection which is hospital acquired pneumonia (HAP). In one aspect of this invention, the combination is used for the treatment of a nosocomial pneumonia infection which is ventilator acquired pneumonia (VAP).

In one embodiment of the invention, the combination of ceftazidime, or a pharmaceutically acceptable salt thereof, and avibactam, or a pharmaceutically acceptable salt thereof, further comprises one or more additional therapeutic agent. In one aspect of this embodiment, the combination further comprises an additional therapeutic agent which is selected from the group consisting of antibacterial agents, beta-lactamase inhibitors and antifungal agents. In one aspect of this embodiment, the combination further comprises an antibacterial agent selected from the group consisting of tobramycin, levofloxacin, vancomyicn, linezolid, tigecycline and colistin.

In one embodiment of the invention, the combination of ceftazidime, or a pharmaceutically acceptable salt thereof, and avibactam, or a pharmaceutically acceptable salt thereof, are administered simultaneously. In another embodiment of the invention, the combination of ceftazidime, or a pharmaceutically acceptable salt thereof, and avibactam, or a pharmaceutically acceptable salt thereof, are independently formulated and co-administered. In another embodiment of the invention, the combination of ceftazidime, or a pharmaceutically acceptable salt thereof, and avibactam, or a pharmaceutically acceptable salt thereof, are independently formulated and administered sequentially. In any of the above embodiments of the invention, the combination comprises about 2000 mg of ceftazidime, or a pharmaceutically acceptable salt thereof, and about 500 mg of avibactam, or pharmaceutically acceptable salt thereof, per dose. In one aspect of these embodiments, the combination is administered approximately every eight hours. In one aspect of any of these embodiments, the combination is administered approximately every twelve hours.

In one embodiment of the invention, combination of ceftazidime, or a pharmaceutically acceptable salt thereof, and avibactam, or a pharmaceutically acceptable salt thereof, is administered intravenously. In one aspect of this embodiment, the combination is administered intravenously over the course of approximately 1 to 2 hours. In one aspect of this embodiment, the combination is administered intravenously over the course of approximately 1 hour. In a different aspect of this [symbol] embodiment, the combination is administered intravenously over the course of approximately 2 hours.

Any of the above embodiments and aspects of embodiments may be combined with any other to form additional intended embodiments of the invention.

Numerous standard references are available that describe procedures for preparing various compositions suitable for administering the compounds according to the invention. Examples of potential compositions and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

The compositions may be solid or liquid and be presented in the pharmaceutical forms, such as for example, plain or sugar-coated tablets, gelatin capsules, granules, suppositories, injectable preparations, ointments, creams, gels, and prepared according to the usual methods. The active ingredient or ingredients can be incorporated with excipients usually employed in these pharmaceutical compositions, such as talc, gum arabic, lactose, starch, magnesium stearate, cocoa butter, aqueous or non-aqueous vehicles, fatty substances of animal or vegetable origin, paraffin derivatives, glycols, various wetting, dispersing or emulsifying agents and preservatives. In one embodiment of the invention, the dose of the combination of ceftazidime and avibactam is administered intravenously.

The compositions may be presented in the form of a lyophilisate intended to be dissolved extemporaneously in an appropriate vehicle, e.g., apyrogenic sterile water. For example, the composition may be formulated as a solid dosage form, such as a dry powder, to be constituted with a diluent before administration. In exemplary embodiments, the composition may be formulated as a dry powder comprising a combination of ceftazidime and avibactam. The dry powder may be constituted with a sterile diluent, such as water, to form a constituted solution before administration. The pH of the constituted solution may be between about 4 and about 10. In other embodiments, the pH of the constituted solution may be between about 5.6 and about 7. The constituted solution can be further diluted before administration using an appropriate solution, such as an infusion solution. Examples of such infusion solutions are 0.9% sodium chloride (normal saline), 5% dextrose, 2.5% dextrose and 0.45% sodium chloride and lactated Ringer's solution.

[symbol]

The compositions may be formulated in various liquid oral dosage forms, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compositions of the present invention may be injected, for example, intravenously, in the form of an isotonic sterile solution. Other preparations are also possible.

In some embodiments, the methods may include administering the combination of ceftazidime and avibactam every 4 hours, 6 hours, 8 hours, 12 hours, 18 hours or every 24 hours. For example, the combination of ceftazidime and avibactam may be administered every 8 hours intravenously by infusion over approximately one hour. For example, the combination of ceftazidime and avibactam may be administered every 8 hours intravenously by infusion over approximately two hours. In other embodiments, the methods may include administering the combination of ceftazidime and avibactam through continuous or prolonged infusion. For example, the combination of ceftazidime and avibactam may be administered by infusion over 3 hours, 4 hours, 5 hours, 6 hours, 7 hours, 8 hours, 9 hours, 10 hours, 11 hours or 12 hours. In other embodiments, the duration of infusion may be more than 12 hours, e.g., 13 hours, 14 hours, 15 hours, 16 hours, 17 hours, 18 hours, 19 hours, 20 hours, 21 hours or 22 hours, 23 hours or 24 hours.

The duration of treatment may depend on the severity infection and the patient's clinical and bacteriological progress, as well as any co-morbidities the patient may have. In some embodiments, the treatment may last between about 5 to 14 days. In other embodiments, the treatment may last between about 5 to 7 days. For example, about the combination of about 2000 mg of ceftazidime and about 500 mg of avibactam may be administered every 8 hours for about five to fourteen days. In further embodiments, about 2000 mg of ceftazidime and about 500 mg of avibactam may be administered every 8 hours for about five to ten days. In other embodiments, about 2000 mg of ceftazidime and about 500 mg of avibactam may be administered every 8 hours for about five to seven days.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per practice in the art. Alternatively, “about” with respect to the compositions can mean plus or minus a range of up to 20%, preferably up to 10%, more preferably up to 5%. Alternatively, particularly with respect to biological systems or processes, the term can mean within an order of magnitude, preferably within 5-fold, and more [symbol] preferably within 2-fold, of a value. Where particular values are described in the application and claims, unless otherwise stated the term “about” means within an acceptable error range for the particular value. For example, when referring to a period of time, e.g., hours, the present values (±20%) are more applicable. Thus, 6 hours can be, e.g., 4.8 hours, 5.5 hours, 6.5 hours, 7.2 hours, as well as the usual 6 hours.

The terms “treat,” “treatment,” and “treating” refer to one or more of the following: relieving or alleviating at least one symptom of a bacterial infection in a subject; relieving or alleviating the intensity and/or duration of a manifestation of bacterial infection experienced by a subject; and arresting, delaying the onset (i.e., the period prior to clinical manifestation of infection) and/or reducing the risk of developing or worsening a bacterial infection.

The term “therapeutically effective” applied to dose or amount refers to that quantity of a compound or pharmaceutical composition that is sufficient to result in a desired activity upon administration to a mammal in need thereof. An “effective amount” means the amount of a compound according to the invention that, when administered to a patient for treating an infection or disease is sufficient to effect such treatment. The “effective amount” will vary depending on the active ingredient, the state of infection, disease or condition to be treated and its severity, and the age, weight, physical condition and responsiveness of the mammal to be treated.

[symbol]

### EXAMPLES

### Example 1

### In Vitro Potency of CAZ-AVI in Pulmonary Surfactant Bacterial Strains

The bacterial strains used in this testing were part of the microbiological culture collection housed at AstraZeneca R&D Boston (AstraZeneca Research Collection, designated ARC). The panel of bacterial isolates used for this testing was comprised of five CLSI QC reference strains and the remainder were either recent clinical isolates expressing β-lactamases or isolates from the primary bacterial screening panels.

### Study Design

MIC values were determined using the CLSI broth microdilution methodology with slight variation. Stock compound mother plates were prepared and used to spot 2 μL aliquots of serial 2-fold drug dilutions to columns 1-11 of 96-well daughter plates using a Perkin-Elmer MiniTrak™ MultiPosition dispenser. Column 12 did not contain drug and served as a growth control. An inoculum volume of 100 μL (5×10E⁵ CFU/mL) in CAMHB containing 0, 1, 2.5, 5, or 10% pulmonary surfactant was added using a multichannel Finnpipette® to each well of the 96-well plate. Avibactam was tested at a fixed concentration of 4 μg/mL when tested in combination with ceftazidime.

### Experimental Procedures

The minimum inhibitory concentration (MIC) values against each organism/drug combination were determined using broth microdilution methodology according to CLSI guidelines. The recommended reference bacterial strains for each test group and reference compounds were incorporated into each test. For Gram-negatives, the reference bacterial strains were Escherichia coli ATCC 25922, E. coli ATCC 35218, Klebsiella pneumoniae ATCC 700603, and Pseudomonas aeruginosa ATCC 27853. Staphylococcus aureus ATCC 29213 was the reference bacterial strain for the Gram-positives.

### Data Analysis

MIC values for individual isolates were read visually. The MICs for each compound or combination in pulmonary surfactant was compared to the compound or combination tested in CAMHB alone.

### Results

The antibacterial activity of ceftazidime, avibactam, ceftazidime-avibactam, and daptomycin tested in varying concentrations of CAMHB is listed in Table 1. Other than an occasional (+/−) 2-fold transient variation in MIC, no MIC increases were observed for ceftazidime, avibactam, or ceftazidime-avibactam against any of the Gram-positive or Gram-negative bacterial strains tested in [symbol] up to 10% pulmonary surfactant. In contrast, daptomycin MICs increased substantially (32- to >128-fold) versus the S. aureus strains tested. As little as 1% pulmonary surfactant resulted in a 32-fold increase in daptomycin S. aureus MICs.

MIC data for ceftazidime, ceftazidime-avibactam and daptomycin versus CLSI QC reference bacterial strains is listed in Error! Reference source not found. MIC values for ceftazidime, ceftazidime-avibactam, and daptomycin were within the CLSI QC ranges for each strain.

### Conclusions

No surfactant-related MIC increases were observed for ceftazidime, avibactam, or ceftazidime-avibactam in the presence of up to 10% pulmonary surfactant against any of the bacterial strains tested. The consistent antibacterial activity of ceftazidime, avibactam, and ceftazidime-avibactam in the presence of varying concentrations of pulmonary surfactant is noteworthy especially for drugs that may be considered for treating respiratory infections. As expected, MICs for the daptomycin positive control increased significantly against S. aureus as the pulmonary surfactant concentration was increased.

TABLE 1
Antibacterial activity of ceftazidime, avibactam, and ceftazidime-avibactam in the
presence of varying concentrations of pulmonary surfactant

| | | | Surfactant | | | | |
|---|---|---|---|---|---|---|---|
| | | Description | Concentration | MIC μg/mL | | | |
| Genus Species | Strain code | (β-lactamase content) | (%) | ceftazidime | avibactam | CAZ-AVIa | daptomycin |
| S. aureus | ARC12 | CLSI QC strain, ATCC 29213 | 0 | 8 | >64 | 8 | 0.5 |
| S. aureus | ARC12 | CLSI QC strain, ATCC 29213 | 1 | 16 | >64 | 8 | 16 |
| S. aureus | ARC12 | CLSI QC strain, ATCC 29213 | 2.5 | 16 | >64 | 16 | 64 |
| S. aureus | ARC12 | CLSI QC strain, ATCC 29213 | 5 | 8 | >64 | 8 | >64 |
| S. aureus | ARC12 | CLSI QC strain, ATCC 29213 | 10 | 8 | >64 | 8 | 64 |
| S. aureus | ARC516 | MRSA, quinolone-resistant | 0 | >64 | >64 | >64 | 0.5 |
| S. aureus | ARC516 | MRSA, quinolone-resistant | 1 | >64 | >64 | >64 | 16 |
| S. aureus | ARC516 | MRSA, quinolone-resistant | 2.5 | >64 | >64 | >64 | 64 |
| S. aureus | ARC516 | MRSA, quinolone-resistant | 5 | >64 | >64 | >64 | >64 |
| S. aureus | ARC516 | MRSA, quinolone-resistant | 10 | >64 | >64 | >64 | >64 |
| E. coli | ARC4 | CLSI QC strain, ATCC 25922 | 0 | 0.25 | 32 | 0.25 | — |
| E. coli | ARC4 | CLSI QC strain, ATCC 25922 | 1 | 0.25 | 32 | 0.12 | — |
| E. coli | ARC4 | CLSI QC strain, ATCC 25922 | 2.5 | 0.25 | 32 | 0.12 | — |
| E. coli | ARC4 | CLSI QC strain, ATCC 25922 | 5 | 0.25 | 16 | 0.12 | — |
| E. coli | ARC4 | CLSI QC strain, ATCC 25922 | 10 | 0.25 | 16 | 0.12 | — |
| E. coli | ARC016 | CLSI QC strain, ATCC 35218 | 0 | 0.12 | 16 | 0.12 | — |
| | | (TEM-1) | | | | | |
| E. coli | ARC016 | CLSI QC strain, ATCC 35218 | 1 | 0.12 | 16 | 0.12 | — |
| | | (TEM-1) | | | | | |
| E. coli | ARC016 | CLSI QC strain, ATCC 35218 | 2.5 | 0.12 | 16 | 0.12 | — |
| | | (TEM-1) | | | | | |
| E. coli | ARC016 | CLSI QC strain, ATCC 35218 | 5 | 0.12 | 16 | 0.12 | — |
| | | (TEM-1) | | | | | |
| E. coli | ARC016 | CLSI QC strain, ATCC 35218 | 10 | 0.12 | 16 | 0.12 | — |
| | | (TEM-1) | | | | | |
| E. coli | ARC523 | W3110 | 0 | 0.5 | >64 | 0.25 | — |
| E. coli | ARC523 | W3110 | 1 | 0.5 | >64 | 0.25 | — |
| E. coli | ARC523 | W3110 | 2.5 | 0.25 | >64 | 0.25 | — |
| E. coli | ARC523 | W3110 | 5 | 0.5 | >64 | 0.25 | — |
| E. coli | ARC523 | W3110 | 10 | 0.5 | >64 | 0.25 | — |
| E. coli | ARC3690 | (CTX-M-15, SHV-12) | 0 | 64 | 16 | 0.12 | — |
| E. coli | ARC3690 | (CTX-M-15, SHV-12) | 1 | 64 | 16 | 0.12 | — |
| E. coli | ARC3690 | (CTX-M-15, SHV-12) | 2.5 | 64 | 16 | 0.12 | — |
| E. coli | ARC3690 | (CTX-M-15, SHV-12) | 5 | 64 | 16 | 0.12 | — |
| E. coli | ARC3690 | (CTX-M-15, SHV-12) | 10 | 64 | 16 | 0.12 | — |
| E. coli | ARC3666 | (CTX-M-15, OXA-1/30, | 0 | 32 | 8 | 0.12 | — |
| | | SHV-31, TEM-1) | | | | | |
| E. coli | ARC3666 | (CTX-M-15, OXA-1/30, | 1 | 32 | 8 | 0.12 | — |
| | | SHV-31, TEM-1) | | | | | |
| E. coli | ARC3666 | (CTX-M-15, OXA-1/30, | 2.5 | 32 | 8 | 0.12 | — |
| | | SHV-31, TEM-1) | | | | | |
| E. coli | ARC3666 | (CTX-M-15, OXA-1/30, | 5 | 32 | 8 | 0.12 | — |
| | | SHV-31, TEM-1) | | | | | |
| E. coli | ARC3666 | (CTX-M-15, OXA-1/30, | 10 | 32 | 8 | 0.12 | — |
| | | SHV-31, TEM-1) | | | | | |
| K. pneumoniae | ARC561 | CLSI QC strain, ATCC 700603 | 0 | 32 | >64 | 0.5 | — |
| | | (SHV-18, OXA-2, OKP-6) | | | | | |
| K. pneumoniae | ARC561 | CLSI QC strain, ATCC 700603 | 1 | 32 | >64 | 0.5 | — |
| | | (SHV-18, OXA-2, OKP-6) | | | | | |
| K. pneumoniae | ARC561 | CLSI QC strain, ATCC 700603 | 2.5 | 32 | >64 | 0.5 | — |
| | | (SHV-18, OXA-2, OKP-6) | | | | | |
| K. pneumoniae | ARC561 | CLSI QC strain, ATCC 700603 | 5 | 32 | 64 | 0.5 | — |
| | | (SHV-18, OXA-2, OKP-6) | | | | | |
| K. pneumoniae | ARC561 | CLSI QC strain, ATCC 700603 | 10 | 32 | 64 | 0.5 | — |
| | | (SHV-18, OXA-2, OKP-6) | | | | | |
| K. pneumoniae | ARC1865 | Primary Screening Panel | | 0.5 | >64 | 0.25 | — |
| | | (Not sequenced) | | | | | |
| K. pneumoniae | ARC1865 | Primary Screening Panel | 1 | 0.25 | >64 | 0.25 | — |
| | | (Not sequenced) | | | | | |
| K. pneumoniae | ARC1865 | Primary Screening Panel | 2.5 | 0.5 | 64 | 0.25 | — |
| | | (Not sequenced) | | | | | |
| K. pneumoniae | ARC1865 | Primary Screening Panel | 5 | 0.5 | >64 | 0.25 | — |
| | | (Not sequenced) | | | | | |
| K. pneumoniae | ARC1865 | Primary Screening Panel | 10 | 0.5 | >64 | 0.25 | — |
| | | (Not sequenced) | | | | | |
| K. pneumoniae | ARC2528 | Clinical isolate | 0 | 1 | 64 | 1 | — |
| | | (Not sequenced) | | | | | |
| K. pneumoniae | ARC2528 | Clinical isolate | 1 | 2 | 64 | 1 | — |
| | | (Not sequenced) | | | | | |
| K. pneumoniae | ARC2528 | Clinical isolate | 2.5 | 1 | 64 | 1 | — |
| | | (Not sequenced) | | | | | |
| K. pneumoniae | ARC2528 | Clinical isolate | 5 | 1 | 64 | 1 | — |
| | | (Not sequenced) | | | | | |
| K. pneumoniae | ARC2528 | Clinical isolate | 10 | 1 | 32 | 1 | — |
| | | (Not sequenced) | | | | | |
| K. pneumoniae | ARC2929 | (KPC-3) | 0 | >64 | 8 | 0.5 | — |
| K. pneumoniae | ARC2929 | (KPC-3) | 1 | >64 | 8 | 0.5 | — |
| K. pneumoniae | ARC2929 | (KPC-3) | 2.5 | >64 | 8 | 0.5 | — |
| K. pneumoniae | ARC2929 | (KPC-3) | 5 | >64 | 8 | 0.5 | — |
| K. pneumoniae | ARC2929 | (KPC-3) | 10 | >64 | 8 | 0.5 | — |
| K. pneumoniae | ARC2945 | (KPC-2, SHV-11, TEM-1, | 0 | 64 | 8 | 0.12 | — |
| | | OXA-9) | | | | | |
| K. pneumoniae | ARC2945 | (KPC-2, SHV-11, TEM-1, | 1 | 64 | 8 | 0.12 | — |
| | | OXA-9) | | | | | |
| K. pneumoniae | ARC2945 | (KPC-2, SHV-11, TEM-1, | 2.5 | 64 | 8 | 0.12 | — |
| | | OXA-9) | | | | | |
| K. pneumoniae | ARC2945 | (KPC-2, SHV-11, TEM-1, | 5 | 64 | 8 | 0.12 | — |
| | | OXA-9) | | | | | |
| K. pneumoniae | ARC2945 | (KPC-2, SHV-11, TEM-1, | 10 | 64 | 8 | 0.12 | — |
| | | OXA-9) | | | | | |
| K. pneumoniae | ARC3713 | (CTX-M-15, OXA-1/30, | 0 | >64 | 16 | 0.25 | — |
| | | SHV-5, TEM-1) | | | | | |
| K. pneumoniae | ARC3713 | (CTX-M-15, OXA-1/30, | 1 | >64 | 16 | 0.25 | — |
| | | SHV-5, TEM-1) | | | | | |
| K. pneumoniae | ARC3713 | (CTX-M-15, OXA-1/30, | 2.5 | >64 | 16 | 0.25 | — |
| | | SHV-5, TEM-1) | | | | | |
| K. pneumoniae | ARC3713 | (CTX-M-15, OXA-1/30, | 5 | >64 | 16 | 0.25 | — |
| | | SHV-5, TEM-1) | | | | | |
| K. pneumoniae | ARC3713 | (CTX-M-15, OXA-1/30, | 10 | >64 | 16 | 0.25 | — |
| | | SHV-5, TEM-1) | | | | | |
| P. aeruginosa | ARC3 | CLSI QC strain, ATCC 27853 | 0 | 1 | >64 | 1 | — |
| P. aeruginosa | ARC3 | CLSI QC strain, ATCC 27853 | 1 | 1 | >64 | 1 | — |
| P. aeruginosa | ARC3 | CLSI QC strain, ATCC 27853 | 2.5 | 2 | >64 | 1 | — |
| P. aeruginosa | ARC3 | CLSI QC strain, ATCC 27853 | 5 | 1 | >64 | 1 | — |
| P. aeruginosa | ARC3 | CLSI QC strain, ATCC 27853 | 10 | 2 | >64 | 1 | — |
| P. aeruginosa | ARC545 | PAO1 | 0 | 1 | >64 | 1 | — |
| P. aeruginosa | ARC545 | PAO1 | 1 | 1 | >64 | 1 | — |
| P. aeruginosa | ARC545 | PAO1 | 2.5 | 1 | >64 | 1 | — |
| P. aeruginosa | ARC545 | PAO1 | 5 | 1 | >64 | 1 | — |
| P. aeruginosa | ARC545 | PAO1 | 10 | 1 | >64 | 1 | — |
| P. aeruginosa | ARC2761 | Clinical isolate | 0 | 2 | >64 | 1 | — |
| P. aeruginosa | ARC2761 | Clinical isolate | 1 | 2 | >64 | 1 | — |
| P. aeruginosa | ARC2761 | Clinical isolate | 2.5 | 1 | >64 | 1 | — |
| P. aeruginosa | ARC2761 | Clinical isolate | 5 | 2 | >64 | 1 | — |
| P. aeruginosa | ARC2761 | Clinical isolate | 10 | 2 | >64 | 1 | — |
| P. aeruginosa | ARC2532 | Clinical isolate | 0 | 2 | >64 | 2 | — |
| P. aeruginosa | ARC2532 | Clinical isolate | 1 | 2 | >64 | 1 | — |
| P. aeruginosa | ARC2532 | Clinical isolate | 2.5 | 2 | >64 | 1 | — |
| P. aeruginosa | ARC2532 | Clinical isolate | 5 | 2 | >64 | 1 | — |
| P. aeruginosa | ARC2532 | Clinical isolate | 10 | 2 | >64 | 1 | — |
| P. aeruginosa | ARC3508 | (derepressed AmpC) | 0 | 32 | >64 | 2 | — |
| P. aeruginosa | ARC3508 | (derepressed AmpC) | 1 | 32 | >64 | 2 | — |
| P. aeruginosa | ARC3508 | (derepressed AmpC) | 2.5 | 32 | >64 | 2 | — |
| P. aeruginosa | ARC3508 | (derepressed AmpC) | 5 | 32 | >64 | 2 | — |
| P. aeruginosa | ARC3508 | (derepressed AmpC) | 10 | 32 | >64 | 2 | — |

^{a}CAZ-AVI = Ceftazidime-avibactam

### Example 2

### Potential Drug Interaction with Other Commonly Co-Administered Agents

A checkerboard assay was used to determine what, if any, interaction between ceftazidime and the ceftazidime-avibactam combination had with six established antibacterial agents: tobramycin, levofloxacin, vancomycin, linezolid, tigecycline and colistin. The MIC of ceftazidime and ceftazidime-avibactam with and without the presence of these antibacterial agents at various concentrations was compared to give a series of fractional inhibitory concentration index (FICI) values. A mean FICI was taken from each combination checkerboard and interpreted according to accepted criteria. Where antibacterial agents had no effect (vancomycin and linezolid against Gram negative isolates; colistin against Gram-positive isolates) the MIC alone of ceftazidime and ceftazidime-avibactam was compared with the MIC in combination with the Cₘₐₓ and 0.5×Cₘₐₓ of these antibacterial agents. Four highly-expressed AmpC, eight extended-spectrum beta-lactamase (ESBL) including two CTX-M-15 and five KPC producing Enterobacteriaceae and P. aeruginosa were included as well as representatives from each species with basal MICs. Three S. aureus and three E. faecalis were also included. No interaction, synergistic or antagonistic, between ceftazidime or ceftazidime-avibactam and any other antibacterial agent was observed. From this experiment, it was concluded that ceftazidime and ceftazidime-avibactam will not interact adversely microbiologically with any of the tested drugs when used in combination.

[symbol]

The stains tested are listed below in Table 2.

TABLE 2

| | | | Reference Number |
|---|---|---|---|
| Isolate Number | Organism | Phenotype | (if applicable) |
| CAZ-AVI-02-075 - 01 | E. cloacae | Basal MICs | |
| CAZ-AVI-02-075 - 02 | E. cloacae | Derepressed AmpC | |
| CAZ-AVI-02-075 - 03 | E. cloacae | Derepressed AmpC | |
| CAZ-AVI-02-075 - 04 | E. coli | Basal MICs | ATCC 25922 |
| CAZ-AVI-02-075 - 05 | E. coli | CTX-M-15 | |
| CAZ-AVI-02-075 - 06 | E. coli | TEM-4 | |
| CAZ-AVI-02-075 - 07 | E. coli | SHV-12 | |
| CAZ-AVI-02-075 - 08 | E. coli | SHV-2 | |
| CAZ-AVI-02-075 - 09 | K. pneumoniae | Basal MICs | |
| CAZ-AVI-02-075 - 10 | K. pneumoniae | TEM-4 | |
| CAZ-AVI-02-075 - 11 | K. pneumoniae | CTX-M-15 | |
| CAZ-AVI-02-075 - 12 | K. pneumoniae | SHV-12 | |
| CAZ-AVI-02-075 - 13 | K. pneumoniae | KPC-3 | |
| CAZ-AVI-02-075 - 14 | K. pneumoniae | KPC-3 | |
| CAZ-AVI-02-075 - 15 | K. pneumoniae | KPC-2 | |
| CAZ-AVI-02-075 - 16 | P. aeruginosa | Basal MICs | ATCC 27853 |
| CAZ-AVI-02-075 - 17 | P. aeruginosa | Derepressed AmpC | |
| CAZ-AVI-02-075 - 18 | P. aeruginosa | Derepressed AmpC | |
| CAZ-AVI-02-075 - 19 | P. aeruginosa | PER | |
| CAZ-AVI-02-075 - 20 | P. aeruginosa | KPC-2 | |
| CAZ-AVI-02-075 - 21 | P. aeruginosa | KPC-2 | |
| CAZ-AVI-02-075 - 22 | S. aureus | Basal MICs | ATCC 29213 |
| CAZ-AVI-02-075 - 23 | S. aureus | Penicillinase Positive | |
| CAZ-AVI-02-075 - 24 | S. aureus | Penicillinase Positive | |
| CAZ-AVI-02-075 - 25 | E. faecalis | Basal MICs | |
| CAZ-AVI-02-075 - 26 | E. faecalis | Basal MICs | |
| CAZ-AVI-02-075 - 27 | E. faecalis | Basal MICs | |

### Supply of Drug Products:

Tobramycin, Levofloxacin, Vancomycin and Colistin were supplied from Sigma-Aldrich (Dorset, UK). Linezolid and Tigecycline were supplied from Molekula (Dorset, UK).

### In Vitro Susceptibility Test Methods:

All MIC determinations including those performed in the checkerboard assay were done in cation-adjusted Mueller-Hinton broth (purchased from Becton Dickinson, Oxford UK).

Initial MIC data were determined by microbroth dilution methods as recommended by CLSI (2012b). Checkerboards were made in accordance with the method given by Pillai et. al., [symbol] Antimicrobial Combinations in Antibiotics, LABORATORY MEDICINE (Lorian Ed., 3^{rd} Ed. (2003)) p. 365-440 as per standard microbroth dilution tests but with agents in combination. Stock solutions were made and serially diluted in cation-adjusted Mueller-Hinton broth with concentrations at four times that required, except ceftazidime-avibactam where both agents were made at eight times the concentration required, to account for the antibacterial dilutions to be made. A final avibactam concentration of 4 mg/L was used throughout the study. Antibacterial agents were combined into microtitre plates using the Evolution III liquid handling system. In the case of Gram-negative isolates against vancomycin and linezolid the MIC alone of ceftazidime and ceftazidime-avibactam was compared with the MIC in the presence of the Cₘₐₓ and 0.5×Cₘₐₓ of vancomycin (Baxter Healthcare Corp., 2008) and linezolid (MacGowan, Pharmacokinetics and Pharmacodynamic Profile of Linezolid in Healthy Volunteers and Patients with Gram-Positive Infections, JAC, 51 (Sup S2):ii17-1125 (2003)).

In the case of Gram-positive isolates against colistin the MIC alone of ceftazidime and ceftazidime-avibactam was compared with the MIC in the presence of the Cₘₐₓ and 0.5×Cₘₐₓ of colistin as referred to in Couet et. al, Clinical Microbiology and Infection, Colistin Pharmacokinetics: the Fog is Lifting, CMI 18:30-39 (2011).

Checkerboard plates were inoculated and incubated in accordance with CLSI guidelines (2012b) at 35±2° C. in ambient air for 16-20 h. MICs were recorded the following day as the lowest concentration or combination of concentrations required to inhibit all visible growth. From the MIC data, FICIs were calculated for each checkerboard according to the method of Meletiadis et. al., Defining Fractional Inhibitory Concentration Index Cutoffs for Additive Interactions Based on Self-Drug Additive Combinations, Monte Carlo Simulation Analysis and in vitro-in vivo Correlation Data for Antifungal Drug Combinations Against Aspergillus fumigatus, AAC 54:602-09 (2010). Each well corresponding to an MIC had an FICI calculated by the following formula:

FICI=FIC_{A}+FIC_{B}=(C_{A/}MIC_{A})+(C_{B}/MIC_{B})

Where MIC_{A} is the MIC of the combination antimicrobial agent alone and MIC_{B} is the MIC of ceftazidime or ceftazidime-avibactam alone. C_{A} is the concentration of the combination drug in combination and C_{B} is the concentration of ceftazidime or ceftazidime-avibactam in combination. As each checkerboard gives a number of FICI values, an arithmetic mean is calculated from all values to give the mean FICI for two agents combined against one isolate. Mean FICIs were interpreted by the following criteria given by Odds (2003) which are now the accepted criteria by most journals:

| | |
|---|---|
| ≦0.5 | Synergy |
| 0.51-4 | Indifference |

[symbol]

4 Antagonism

The wide FICI range for interpreting indifferent interactions is due to the inherent variability of MIC results in doubling dilution schemes (Pillai et al. 2005). Meletiadis et al. (2010) suggest that an FICI of greater than 2 should be interpreted as antagonistic but that such interpretations should be treated with caution due to this variability.

### Results

Summary MIC data are shown in Table 3. Summary mean FICI data are shown in Table 4. Ceftazidime/ceftazidime-avibactam MIC ratios alone and in combination where FICI calculations are not possible are shown in Table 5.

### Ceftazidime

The mean FICI range for ceftazidime combined with a second agent for all antibacterial agents, where it was calculated, was 0.64 to 1.99.

There were no cases of a mean FICI of greater than 2 in all combinations with ceftazidime. Where the Cₘₐₓ and 0.5×Cₘₐₓ of vancomycin and linezolid was combined with ceftazidime against Gram-negative isolates, the MIC of ceftazidime alone and in combination remained within one doubling dilution in all cases. Similarly where the Cₘₐₓ and 0.5×Cₘₐₓ of colistin was combined with ceftazidime against Gram-positive isolates, the MIC of ceftazidime alone and in combination remained within one doubling dilution in all cases.

### Ceftazidime-Avibactam

The mean FICI range for ceftazidime-avibactam combined with all antibacterial agents was 0.72 to 2.13.

Ceftazidime-avibactam in combination with colistin against K. pneumoniae 012 gave a mean FICI of 2.13. This was the only example of a mean FICI of greater than 2 for all combinations with ceftazidime-avibactam.

Where the Cₘₐₓ and 0.5×Cₘₐₓ of vancomycin and linezolid was combined with ceftazidime-avibactam against Gram-negative isolates, the MIC of ceftazidime-avibactam alone and in combination remained within one doubling dilution in all cases except one. In this case (E. coli 08) the ceftazidime-avibactam MIC reduced from 0.12 mg/L to 0.03 mg/L when in combination with the Cₘₐₓ of vancomycin. Where the Cₘₐₓ and 0.5×Cₘₐₓ of colistin was combined with ceftazidime-avibactam against Gram-positive isolates, the MIC of ceftazidime-avibactam alone and in combination remained within one doubling dilution in all cases.

TABLE 3
Summary MIC data for all antibacterial agents against all isolates.
Antibacterial-isolate combinations with no activity are not included.

| | | | MIC (mg/L) | | | | | | | |
|---|---|---|---|---|---|---|---|---|---|---|
| Isolate | | | | Ceftazidime- | | | | | | |
| Number | Species | Phenotype | Ceftazidime | avibactam | Tobramycin | Levofloxacin | Vancomycin | Linezolid | Tigecycline | Colist [symbol] |
| 1 | E. cloacae | Basal MICs | 0.12 | 0.06 | 0.25 | 0.03 | | | 0.5 | 0.25 |
| 2 | E. cloacae | Derepressed | 256 | 2 | 0.25 | 0.5 | | | 1 | 0.25 |
| | | AmpC | | | | | | | | |
| 3 | E. cloacae | Derepressed | 128 | 1 | 0.25 | 0.06 | | | 0.5 | 0.25 |
| | | AmpC | | | | | | | | |
| 4 | E. coli | Basal MICs | 0.25 | 0.12 | 0.5 | 0.015 | | | 0.12 | 0.25 |
| 5 | E. coli | CTX-M-15 | 64 | 0.12 | 64 | 16 | | | 0.25 | 0.5 |
| 6 | E. coli | TEM-4 | 8 | 0.06 | 1 | 0.015 | | | 0.5 | 0.25 |
| 7 | E. coli | SHV-12 | 16 | 0.06 | 1 | 0.015 | | | 0.5 | 0.5 |
| 8 | E. coli | SHV-2 | 1 | 0.06 | 64 | 16 | | | 0.25 | 0.25 |
| 9 | K. pneumoniae | Basal MICs | 0.12 | 0.12 | 0.25 | 0.03 | | | 0.25 | 0.25 |
| 10 | K. pneumoniae | TEM-4 | 32 | 0.25 | 4 | 0.03 | | | 0.25 | 0.5 |
| 11 | K. pneumoniae | CTX-M-15 | 32 | 0.25 | 0.25 | 16 | | | 1 | 0.25 |
| 12 | K. pneumoniae | SHV-12 | 64 | 0.12 | 0.25 | 0.03 | | | 0.5 | 0.25 |
| 13 | K. pneumoniae | KPC-3 | 128 | 1 | 0.25 | 0.06 | | | 1 | 0.25 |
| 14 | K. pneumoniae | KPC-3 | 16 | 0.25 | 8 | 16 | | | 0.25 | 0.25 |
| 15 | K. pneumoniae | KPC-2 | 64 | 2 | 0.12 | 0.06 | | | 0.5 | 0.25 |
| 16 | P. aeruginosa | Basal MICs | 1 | 1 | 0.25 | 1 | | | 16 | 1 |
| 17 | P. aeruginosa | Derepressed | 8 | 8 | 0.5 | 8 | | | 32 | 0.5 |
| | | AmpC | | | | | | | | |
| 18 | P. aeruginosa | Derepressed | 64 | 4 | 0.5 | 0.12 | | | 32 | 1 |
| | | AmpC | | | | | | | | |
| 19 | P. aeruginosa | PER | 128 | 16 | 1 | 16 | | | 32 | 1 |
| 20 | P. aeruginosa | KPC-2 | 128 | 8 | 256 | 16 | | | 16 | 1 |
| 21 | P. aeruginosa | KPC-2 | 64 | 2 | 8 | 32 | | | 2 | 2 |
| 22 | S. aureus | Basal MICs | 8 | 8 | 0.25 | 0.25 | 1 | 2 | 0.12 | |
| 23 | S. aureus | Basal MICs, | 16 | 16 | 0.5 | 0.12 | 1 | 2 | 0.12 | |
| | | Penicillinase+ | | | | | | | | |
| 24 | S. aureus | Basal MICs, | 8 | 8 | 0.25 | 0.12 | 1 | 2 | 0.12 | |
| | | Penicillinase+ | | | | | | | | |
| 25 | E. faecalis | Basal MICs | >512 | >512 | 16 | 0.5 | 1 | 1 | 0.06 | |
| 26 | E. faecalis | Basal MICs | 256 | 256 | 16 | 1 | 2 | 2 | 0.06 | |
| 27 | E. faecalis | Basal MICs | 256 | 256 | 8 | 1 | 2 | 2 | 0.06 | |

[symbol]

indicates data missing or illegible when filed

TABLE 4
Mean FICI summary for all combinations (where appropriate) FICI values were not calculated
for assays involving antibacterial-isolate combinations with no activity

| | | | Ceftazidime | | | | | | Ceftazidime-avibactam | | | | | |
|---|---|---|---|---|---|---|---|---|---|---|---|---|---|---|
| Isolate Number | Species | Phenotype | Tob | Lev | Van | Lzd | Tig | Col | Tob | Lev | Van | Lzd | Tig | Co [symbol] |
| 1 | E. cloacae | Basal MICs | 1.28 | 1.33 | | | 1.24 | 1.25 | 1.03 | 1.17 | | | 1.24 | 1. [symbol] |
| 2 | E. cloacae | Derepressed AmpC | 1.17 | 0.99 | | | 0.93 | 1.54 | 1.28 | 1.24 | | | 1.67 | 1. [symbol] |
| 3 | E. cloacae | Derepressed AmpC | 0.73 | 0.81 | | | 1.05 | 1.12 | 0.98 | 1.23 | | | 0.99 | 1. [symbol] |
| 4 | E. coli | Basal MICs | 1.17 | 1.17 | | | 1.41 | 1.35 | 1.14 | 1.39 | | | 1.25 | 1. [symbol] |
| 5 | E. coli | CTX-M-15 | 1.05 | 1.05 | | | 1.12 | 1.23 | 1.14 | 1.12 | | | 1.77 | 1.58 |
| 6 | E. coli | TEM-4 | 0.99 | 1.05 | | | 1.27 | 1.26 | 1.34 | 1.06 | | | 1.82 | 1.45 |
| 7 | E. coli | SHV-12 | 1.19 | 1.18 | | | 0.95 | 1.66 | 1.74 | 1.16 | | | 1.96 | 1.24 |
| 8 | E. coli | SHV-2 | 0.91 | 1.11 | | | 1.10 | 1.54 | 0.76 | 1.72 | | | 1.89 | 1.78 |
| 9 | K. pneumoniae | Basal MICs | 1.55 | 0.96 | | | 1.16 | 1.25 | 1.13 | 1.65 | | | 1.12 | 1.17 |
| 10 | K. pneumoniae | TEM-4 | 0.64 | 1.13 | | | 1.32 | 0.85 | 1.35 | 1.26 | | | 1.60 | 1.60 |
| 11 | K. pneumoniae | CTX-M-15 | 1.05 | 1.21 | | | 0.94 | 1.54 | 1.04 | 1.21 | | | 1.60 | 1.48 |
| 12 | K. pneumoniae | SHV-12 | 1.04 | 1.16 | | | 0.97 | 1.40 | 1.31 | 1.66 | | | 1.14 | 2.13 |
| 13 | K. pneumoniae | KPC-3 | 1.12 | 1.24 | | | 1.99 | 1.26 | 1.05 | 1.21 | | | 1.79 | 1.23 |
| 14 | K. pneumoniae | KPC-3 | 0.91 | 1.26 | | | 0.74 | 1.05 | 1.23 | 1.23 | | | 1.87 | 1.42 |
| 15 | K. pneumoniae | KPC-2 | 1.05 | 1.26 | | | 1.23 | 1.25 | 1.18 | 1.14 | | | 1.24 | 1.24 |
| 16 | P. aeruginosa | Basal MICs | 1.11 | 1.19 | | | 1.05 | 1.24 | 1.23 | 1.19 | | | 1.24 | 1.24 |
| 17 | P. aeruginosa | Derepressed AmpC | 1.05 | 1.10 | | | 1.23 | 1.21 | 0.98 | 1.12 | | | 1.23 | 1.05 |
| 18 | P. aeruginosa | Derepressed AmpC | 0.89 | 1.21 | | | 1.15 | 1.23 | 0.81 | 1.41 | | | 0.96 | 1.21 |
| 19 | P. aeruginosa | PER | 0.70 | 0.99 | | | 1.13 | 1.43 | 0.73 | 0.94 | | | 1.10 | 1.09 |
| 20 | P. aeruginosa | KPC-2 | 0.97 | 1.13 | | | 1.40 | 1.21 | 0.91 | 1.13 | | | 1.26 | 1.21 |
| 21 | P. aeruginosa | KPC-2 | 0.97 | 1.12 | | | 1.23 | 1.34 | 0.72 | 1.60 | | | 1.57 | 1.82 |
| 22 | S. aureus | Basal MICs | 1.60 | 0.98 | 1.23 | 1.43 | 1.13 | | 1.23 | 1.21 | 0.85 | 1.21 | 1.12 | |
| 23 | S. aureus | Basal MICs, | 0.86 | 1.46 | 0.99 | 1.10 | 1.27 | | 0.99 | 1.32 | 0.99 | 1.10 | 1.48 | |
| | | Penicillinase+ | | | | | | | | | | | | |
| 24 | S. aureus | Basal MICs, | 0.84 | 1.21 | 1.24 | 1.32 | 1.11 | | 1.12 | 1.21 | 0.78 | 1.21 | 1.35 | |
| | | Penicillinase + | | | | | | | | | | | | |
| 25 | E. faecalis | Basal MICs | 0.84 | 1.15 | 1.63 | 1.72 | 1.19 | | 0.84 | 0.99 | 1.52 | 1.81 | 1.14 | |
| 26 | E. faecalis | Basal MICs | 0.74 | 1.20 | 1.18 | 0.99 | 0.73 | | 0.80 | 1.19 | 0.89 | 1.24 | 1.05 | |
| 27 | E. faecalis | Basal MICs | 0.99 | 0.78 | 1.38 | 1.64 | 1.16 | | 0.99 | 1.11 | 0.94 | 0.87 | 1.08 | |
| | | Mean FICI range | 0.64 | 0.78 | 0.99 | 0.99 | 0.73 | 0.85 | 0.72 | 0.94 | 0.78 | 0.87 | 0.96 | 1. [symbol] |
| | | low: | | | | | | | | | | | | |
| | | Mean FICI range | 1.60 | 1.33 | 1.63 | 1.64 | 1.99 | 1.66 | 1.74 | 1.72 | 1.52 | 1.81 | 1.96 | 2. [symbol] |
| | | high: | | | | | | | | | | | | |

[symbol]

indicates data missing or illegible when filed

TABLE 5
MIC ratio of ceftazidime/ceftazidime-avibactam with and without the presence of the 0.5 × Cₘₐₓ and Cₘₐₓ
of vancomycin and linezolid against Gram-negative isolates and colistin against Gram-positive isolates

| | | | Ceftazidime | | | | | | Ceftazidime-avibactam | | | | | |
|---|---|---|---|---|---|---|---|---|---|---|---|---|---|---|
| | | | Van | | Lzd | | Col | | Van | | Lzd | | Col | |
| Isolate | | | 0.5 × | | 0.5 × | | 0.5 × | | 0.5 × | | 0.5 × | | 0.5 × | |
| Number | Species | Phenotype | Cmax | Cmax | Cmax | Cmax | Cmax | Cmax | Cmax | Cmax | Cmax | Cmax | Cmax | Cm [symbol] |
| 1 | E. cloacae | Basal MICs | 1 | 1 | 2 | 1 | | | 1 | 0.5 | 1 | 1 | | |
| 2 | E. cloacae | Derepressed AmpC | 2 | 2 | 1 | 1 | | | 1 | 1 | 1 | 1 | | |
| 3 | E. cloacae | Derepressed AmpC | 1 | 2 | 1 | 1 | | | 0.5 | 0.5 | 1 | 1 | | |
| 4 | E. coli | Basal MICs | 1 | 1 | 1 | 1 | | | 1 | 0.5 | 1 | 0.5 | | |
| 5 | E. coli | CTX-M-15 | 1 | 1 | 1 | 1 | | | 1 | 1 | 1 | 1 | | |
| 6 | E. coli | TEM-4 | 1 | 1 | 1 | 1 | | | 1 | 0.5 | 2 | 2 | | |
| 7 | E. coli | SHV-12 | 1 | 1 | 1 | 0.5 | | | 2 | 1 | 2 | 2 | | |
| 8 | E. coli | SHV-2 | 1 | 2 | 1 | 1 | | | 0.5 | 0.25 | 2 | 1 | | |
| 9 | K. pneumoniae | Basal MICs | 2 | 1 | 1 | 1 | | | 1 | 0.5 | 1 | 1 | | |
| 10 | K. pneumoniae | TEM-4 | 1 | 2 | 1 | 1 | | | 1 | 1 | 1 | 1 | | |
| 11 | K. pneumoniae | CTX-M-15 | 2 | 2 | 1 | 0.5 | | | 1 | 1 | 1 | 1 | | |
| 12 | K. pneumoniae | SHV-12 | 1 | 1 | 1 | 1 | | | 1 | 1 | 1 | 1 | | |
| 13 | K. pneumoniae | KPC-3 | 2 | 1 | 2 | 1 | | | 1 | 1 | 1 | 1 | | |
| 14 | K. pneumoniae | KPC-3 | 2 | 1 | 1 | 1 | | | 2 | 2 | 1 | 2 | | |
| 15 | K. pneumoniae | KPC-2 | 2 | 2 | 1 | 1 | | | 1 | 1 | 1 | 1 | | |
| 16 | P. aeruginosa | Basal MICs | 1 | 0.5 | 1 | 1 | | | 1 | 1 | 1 | 1 | | |
| 17 | P. aeruginosa | Derepressed AmpC | 1 | 1 | 1 | 1 | | | 1 | 1 | 1 | 1 | | |
| 18 | P. aeruginosa | Derepressed AmpC | 1 | 1 | 1 | 1 | | | 1 | 1 | 1 | 1 | | |
| 19 | P. aeruginosa | PER | 1 | 1 | 1 | 1 | | | 2 | 1 | 2 | 1 | | |
| 20 | P. aeruginosa | KPC-2 | 1 | 1 | 1 | 1 | | | 0.5 | 1 | 1 | 1 | | |
| 21 | P. aeruginosa | KPC-2 | 2 | 2 | 2 | 2 | | | 1 | 1 | 1 | 1 | | |
| 22 | S. aureus | Basal MICs | | | | | 1 | 1 | | | | | 1 | 1 |
| 23 | S. aureus | Basal MICs, | | | | | 2 | 2 | | | | | 1 | 1 |
| | | Penicillinase+ | | | | | | | | | | | | |
| 24 | S. aureus | Basal MICs, | | | | | 1 | 1 | | | | | 1 | 1 |
| | | Penicillinase+ | | | | | | | | | | | | |
| 25 | E. faecalis | Basal MICs | | | | | 1 | 1 | | | | | 1 | 1 |
| 26 | E. faecalis | Basal MICs | | | | | 2 | 2 | | | | | 2 | 2 |
| 27 | E. faecalis | Basal MICs | | | | | 2 | 1 | | | | | 2 | 1 |

Ratio calculated by dividing MIC of caz/caz-avi in combination with MIC of caz/caz-avi alo [symbol] [symbol]
indicates data missing or illegible when filed

[symbol]

### Example 3

### Penetration of CAZ-AVI into ELF

Pharmacokinetic studies were carried out to describe the pulmonary disposition of ceftazidime-avibactam within infected and uninfected mice. Then, efficacy studies of ceftazidime and ceftazidime-avibactam against Pseudomonas aeruginosa isolates were undertaken using the neutropenic lung infection model. Between infected and uninfected mice, there were no pharmacokinetic differences observed in the serum or ELF. Using human simulated serum doses of ceftazidime 2000 mg and avibactam 500 mg as a 2 h infusion, maximal activity was noted against those isolates with MICs of 32 μg/mL, where ELF fT>MIC≧19% for the upper 95% confidence interval. Given the MIC₉₀ for ceftazidime-avibactam is 8 μg/mL, there are few isolates with MICs higher and even fewer that are able to grow within the murine lung infection model. As such, ceftazidime directed ELF fT>MIC studies were conducted and showed activity against MICs of 32 μg/mL, where ELF fT>MIC was 12%.

### Neutropenic Lung Infection Model

Pathogen-free, female ICR mice weighing approximately 25 g were acquired from Harlan Sprague Dawley, Inc. (Indianapolis, Ind.) and utilized throughout these experiments. Animals were maintained and used in accordance to National Research Council recommendations, and provided food and water ad libitum. Mice were rendered neutropenic with 100 and 250 mg/kg intraperitoneal injections of cyclophosphamide (Cytoxan®; Bristol-Myers Squibb, Princeton, N.J.) given one and four days prior to inoculation, respectively. Three days prior to inoculation, mice were also given a single 5 mg/kg intraperitoneal injection of uranyl nitrate. This produces a predictable degree of renal impairment to slow drug clearance. Two hours prior to the initiation of antimicrobial therapy, mice were lightly anesthetized using isofluorane (2.5% v/v in 100% oxygen carrier) until the respiratory rate decreased upon visual inspection. Pneumonia was induced by the instillation of 0.05 mL of a 10⁷ CFU inoculum of the test isolate suspended in 3% mucin in normal saline. While the mouse is anesthetized, the inoculum is delivered into the animal's oral cavity, blocking the nares and holding the mouse in a vertical position. Aspiration of bacteria into the lungs occurred as the animals began to spontaneously respire. After allowing full recovery from anesthesia in an oxygen-enriched chamber, inoculated mice were randomized into control groups (0 h and 24 h) and treatment groups (CAZ and CAZ-AVI).

### Characterization of Epithelial Lining Fluid (ELF) Concentrations for Ceftazidime-Avibactam

In these studies, we utilized the previously determined dosing regimen described above. Confirmatory serum pharmacokinetic studies were undertaken in infected mice. For these studies, infected neutropenic mice were dosed with the above calculated regimen and groups of six mice [symbol] were euthanized at multiple time points throughout a 24 h period to confirm target exposures blood was collected via cardiac puncture and the serum samples were stored at −80° C. until analysis. Pharmacokinetic studies were undertaken to describe the epithelial lining fluid concentrations in infected mice. For these studies, infected neutropenic mice were dosed with the above calculated regimen and groups of six mice were euthanized at multiple time points throughout the third dosing period (i.e. 16-24 h). Once euthanized, serum and BAL samples were collected as described above. Serum and BAL samples were stored at −80° C. until analysis. Utilizing the BAL concentration time-profile, the ELF fT>MIC was calculated including the upper 95% confidence interval.

### Characterization of Epithelial Lining Fluid (ELF) Concentrations for Ceftazidime

We utilized a previously determined dosing regimen, in mice, that simulated the serum fT>MIC observed in man given ceftazidime 2000 mg every 8 hours as a 2 hour infusion (8). Confirmatory pharmacokinetic studies were undertaken in infected mice and pharmacodynamic analyses and an assessment of ELF fT>MIC was made from the resulting concentration-time profile. For these studies, infected neutropenic mice were dosed with the above calculated regimen and groups of six mice were euthanized at multiple time points throughout the third dosing interval (i.e. 16-24 h) to confirm target exposures.

Pharmacokinetic studies were undertaken to describe the epithelial lining fluid concentrations in infected mice. For these studies, infected neutropenic mice were dosed with the above calculated regimen and groups of six mice were euthanized at multiple time points throughout the third dosing period (i.e. 16-24 h). Once euthanized, serum and BAL samples were collected as described above. Serum and BAL samples were stored at −80° C. until analysis. Utilizing the BAL concentration time-profile, the ELF fT>MIC was calculated including the upper 95% confidence interval.

Determination of Ceftazidime Dosing Regimen for Directed ELF fT>MIC Studies

It has been observed in surveillance studies that the MIC₉₀ for ceftazidime-avibactam against P. aeruginosa is 8 μg/mL (10-12). Given this, there are few isolates with higher MICs and the number that would grow within the murine lung infection model is even less. Previous literature has also found blood fT>MIC efficacy targets derived for ceftazidime-avibactam against P. aeruginosa correlated well with those obtained for ceftazidime monotherapy, as well as other cephalosporins. Given relative MICs for ceftazidime are readily ≧32 μg/mL, we planned to use the humanized ceftazidime regimen described above to provide further insight into the pharmacodynamic break between efficacy and bacterial growth. It was observed during the experiments the ELF fT>MIC was significantly more than expected and produced ELF fT>MIC that did not allow us to differentiate a needed fT>MIC for efficacy. As such, these studies were conducted to design a regimen in mice that resulted in sufficiently low ELF fT>MIC so that we were able to observe a [symbol] loss of efficacy within the lung infection model. Doses of the previous humanized regimen described above were decreased throughout the dosing period and further pharmacokinetic studies were performed. In these studies, mice were dosed a regimen of ceftazidime over a period of 24 h and sampling took place during the last of three intervals (i.e. 16-24 h). Mice were euthanized and serum and BAL samples were collected, as described above, at predetermined times.

### In Vivo Efficacy: Ceftazidime-Avibactam Regimen

For each of the 28 P. aeruginosa isolates, groups of mice were administered regimens of ceftazidime-avibactam beginning two hours after the initiation of infection. All doses were administered as 0.2 mL subcutaneous injections and consisted of three 8-hour dosing intervals (i.e. 24 hours). To serve as control animals, an additional group of mice were administered normal saline at the same volume, route, and frequency as the treatment regimen. Lungs from all animals were harvested 24 hours after the initiation of therapy; mice that failed to survive for 24 hours were harvested at the time of expiration. The harvesting procedure for all study mice began with euthanization by CO₂ exposure followed by cervical dislocation. After sacrifice, lungs were removed and individually homogenized in normal saline. Serial dilutions of the lung homogenate were plated on trypticase soy agar with 5% sheep blood for CFU determination. In addition to the above mentioned treatment and control groups, another group of 6 infected, untreated mice were harvested at the initiation of dosing and served as 0 h controls. Efficacy, designated as the change in bacterial density, was calculated as the change in log₁₀ bacterial CFU obtained for treated mice after 24 h from that of that starting densities observed in 0 h control animals. The development of resistance was also tested by plating a direct inoculum of the homogenate onto plates containing 32-4 μg/mL of ceftazidime-avibactam.

### In Vivo Efficacy: Ceftazidime Regimen

Nine P. aeruginosa isolates were tested against this ceftazidime regimen previous described (8). Groups of mice were administered the ceftazidime regimen beginning two hours after the initiation of infection. All doses were administered as 0.2 mL subcutaneous injections and consisted of three 8-hour dosing intervals (i.e. 24 hours). To serve as control animals, an additional group of mice were administered normal saline at the same volume, route, and frequency as the treatment regimen. Harvesting and processing of lungs from each animal was done as previously described and efficacy was defined as the change in bacterial density, calculated as the change in log₁₀ bacterial CFU obtained for treated mice after 24 h from that of that starting densities observed in 0 h control animals.

In Vivo Efficacy: Ceftazidime Directed ELF fT>MIC Studies

For these studies, 9 P. aeruginosa isolates were evaluated against the CAZ regimen in mice that yielded specific ELF fT>MIC. Doses were initiated 2 hours after inoculation of the test organisms [symbol] and all doses were administered as 0.2 mL subcutaneous injections and consisted of three 8 hour dosing intervals (i.e. 24 hours). To serve as control animals, an additional group of mice were administered normal saline at the same volume, route, and frequency as the treatment regimen. The harvesting procedure for all study mice began with euthanization by CO₂ exposure followed by cervical dislocation. After sacrifice, lungs were removed and individually homogenized in normal saline. Serial dilutions of the lung homogenate were plated on trypticase soy agar with 5% sheep blood for CFU determination. In addition to the above mentioned treatment and control groups, another group of 6 infected, untreated mice were harvested at the initiation of dosing and served as 0 h controls. Efficacy, designated as the change in bacterial density, was calculated as the change in log₁₀ bacterial CFU obtained for treated mice after 24 h from that of that starting densities observed in 0 h control animals.

### Antibacterials:

Commercially available ceftazidime (Fortaz®, Lot: L716, GlaxoSmithKline Research Triangle Park, N.C., USA) was obtained from the Hartford Hospital Pharmacy Department and utilized for all in vivo studies, Analytical grade avibactam was made by AstraZeneca Pharmaceuticals (Waltham, Mass., USA). Clinical vials of ceftazidime were reconstituted as described in the prescribing information and diluted as appropriate to achieve the desired concentrations; analytical avibactam powders were weighed in a quantity sufficient to achieve the required concentrations and reconstituted immediately prior to use.

### Results

### Bacterial Isolates

The ceftazidime and ceftazidime-avibactam MICs for the 28 isolates included in the efficacy studies are shown in Table 6.

TABLE 6
Phenotypic data for the 29 Gram-negative isolates utilized during
in vivo efficacy studies.

| P. aeruginosa | MIC, μg/mL | | Study | | |
|---|---|---|---|---|---|
| CAIRD # | CAZ-AVI | CAZ | CAZ | CAZ fT > MIC | CAZ-AVI |
| PSA 22 | 4 | 64 | x | | x |
| PSA 971 | 4 | 16 | | | x |
| PSA 1383 | 4 | 64 | | x | x |
| PSA 1384 | 4 | 64 | | x | x |
| PSA 37-8 | 4 | 64 | x | | x |
| PSA 856 | 8 | 8 | | | x |
| PSA 1382 | 8 | 128 | | x | x |
| PSA 1386 | 8 | 128 | | x | x |
| PSA 1387 | 8 | 32 | | x | x |
| PSA 1388 | 8 | 128 | | x | x |
| PSA 1389 | 8 | 64 | | x | x |
| PSA 4-32 | 8 | 32 | x | | x |
| PSA 4-39 | 8 | 32 | | | x |
| PSA 2-69 | 8 | 64 | | | x |
| PSA 8-16 | 8 | 64 | x | | X |
| PSA 24-2 | 8 | 32 | | | X |
| PSA 28-19 | 8 | 128 | x | | X |
| PSA 968 | 16 | 32 | | x | X |
| PSA 1391 | 16 | 128 | | | X |
| PSA 1394 | 16 | 64 | | x | X |
| PSA 4-36 | 16 | 64 | x | | x |
| PSA 4-84 | 16 | 64 | x | | x |
| PSA 1-25 | 16 | 128 | x | | x |
| PSA 1-29 | 16 | 32 | | | x |
| PSA 3-9 | 16 | >128 | x | | x |
| PSA 11-54 | 32 | 32 | | | x |
| PSA 7-6 | 32 | 128 | | | x |
| PSA 14-28 | 64 | 128 | | | x |

CAZ: ceftazidime;
CAZ-AVI: ceftazidime-avibactam;
fT > MIC: directed ELF fT > MIC studies;

[symbol]

### Determination of Host Infection Status on Pharmacokinetic Profiles

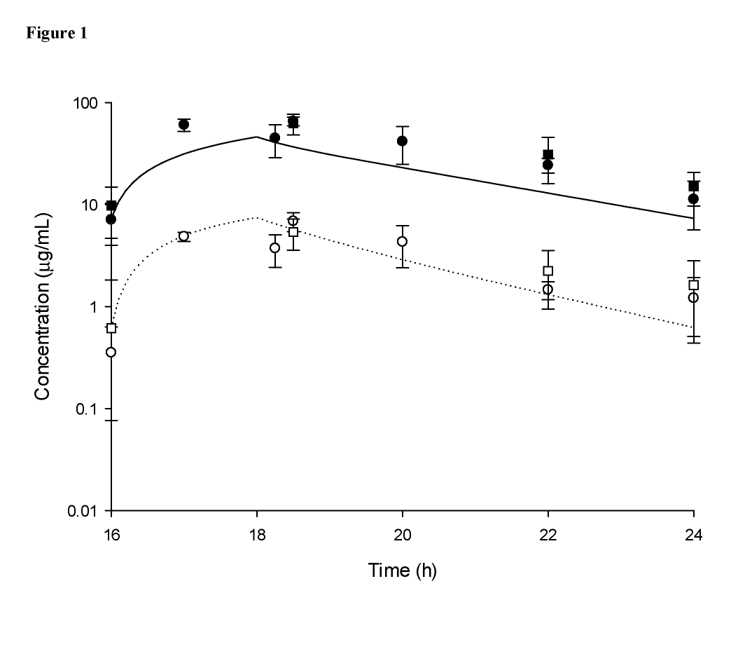

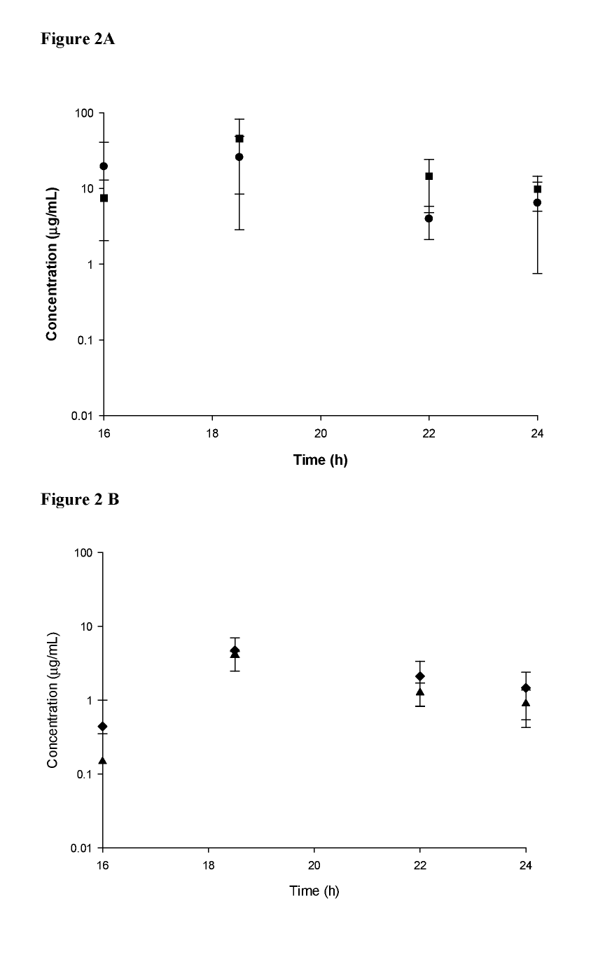

The free drug serum and ELF concentration-time profiles determined in vivo for ceftazidime-avibactam in infected and uninfected mice are shown in FIG. 1 and FIG. 2. The penetration ratios for infected and uninfected mice were similar to each other as described in Table 7. Further, the calculated pharmacodynamic principle, fT>MIC was comparable between infected and uninfected mice (Table 8).

TABLE 7
Epithelial lining fluid (ELF) point penetration ratios for ceftazidime
and avibactam over the third interval of dosing (i.e. 16-24 h) compared
with serum concentrations in infected and uninfected mice.
ELF Point Penetration Ratios for Ceftazidime and Avibactam

| Time | Ceftazidime - Infected | Ceftazidime - Uninfected |
|---|---|---|
| (h) | Penetration Ratio | Penetration Ratio |
| 16 | 2.13 | 1.02 |
| 18.5 | 0.8 | 0.8 |
| 22 | 0.3 | 0.51 |
| 24 | 0.95 | 0.64 |
| Time | Avibactam - Infected | Avibactam - Uninfected |
| (h) | Penetration Ratio | Penetration Ratio |
| 16 | 0.59 | 0.86 |
| 18.5 | 0.66 | 0.74 |
| 22 | 0.69 | 0.93 |
| 24 | 0.8 | 0.95 |

TABLE 8
Epithelial lining fluid (ELF) and serum pharmacodynamic estimates
for ceftazidime and avibactam after human simulated serum doses of
ceftazidime-avibactam 2000-500 mg every 8 h as a 2 h infusion in man
as compared with that observed in infected and uninfected mice.
Pharmacodynamic Estimates for Ceftazidime and Avibactam

| | |
|---|---|
| Ceftazidime - | Ceftazidime - |
| Infected | Uninfected |

| fT > MIC | ELF | Serum | ELF | Serum |
|---|---|---|---|---|
| 4 | 100% | 100% | 100% | 100% |
| 8 | 88% | 88% | 97% | 100% |
| 16 | 63% | 75% | 63% | 78% |
| 32 | 0% | 50% | 34% | 62% |
| 64 | 0% | 0% | 0% | 0% |

| | |
|---|---|
| Avibactam - | Avibactam - |
| Infected | Uninfected |

| fT > MIC | ELF | Serum | ELF | Serum |
|---|---|---|---|---|
| 1 | 84% | 88% | 88% | 88% |
| 2 | 50% | 56% | 75% | 75% |
| 4 | 3% | 34% | 19% | 19% |
| 8 | 0% | 0% | 0% | 0% |

### Characterization of Epithelial Lining Fluid (ELF) Concentrations for Ceftazidime-Avibactam

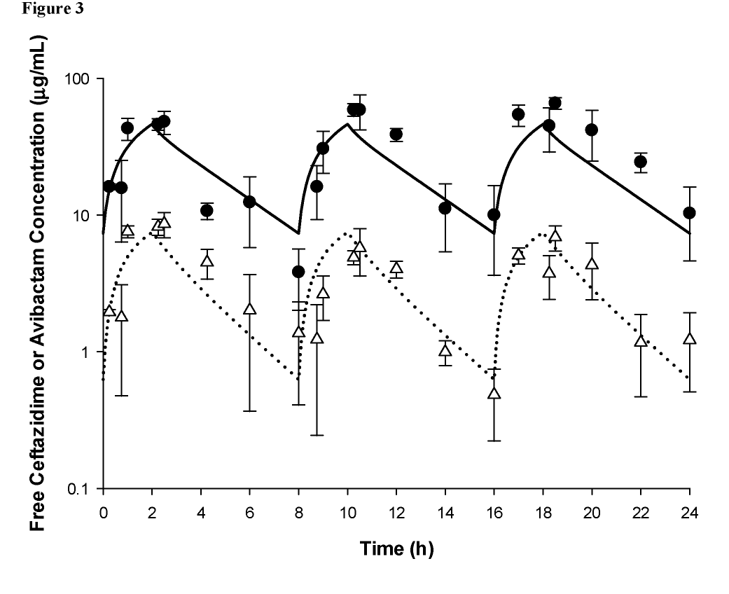

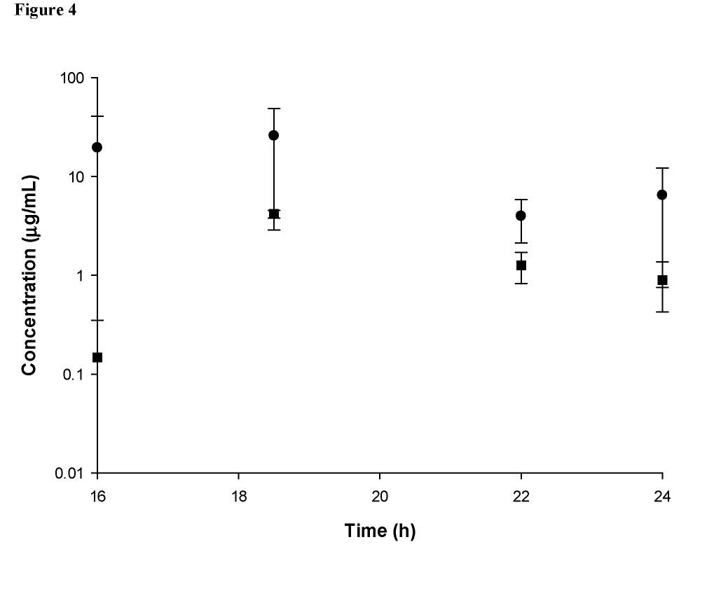

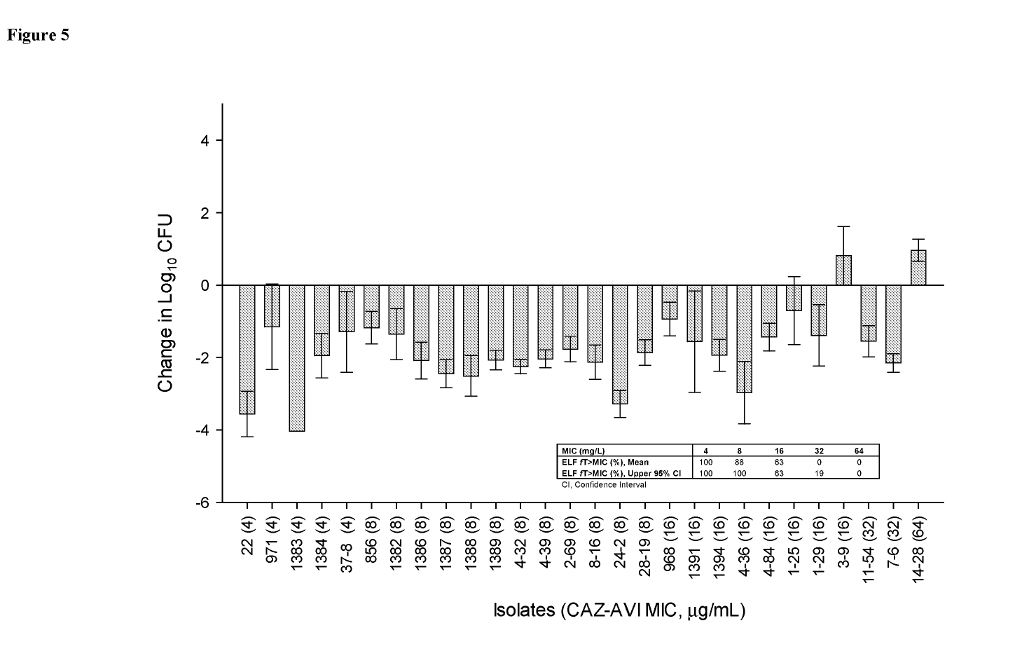

The free drug serum pharmacokinetic profiles determined in vivo for ceftazidime-avibactam are shown in FIG. 3. It is clear from these figures that the murine serum exposure profiles are similar to those observed in humans, importantly, all the fT>MIC in serum attained for these regimens across the range of MICs tested were comparable. The ELF concentrations over the third dosing interval are shown in FIG. 4. From these data, fT>MIC values were determined against a range of MICs. The mean and upper 95% confidence interval are described in FIG. 5.

### Characterization of Epithelial Lining Fluid (ELF) Concentrations for Ceftazidime

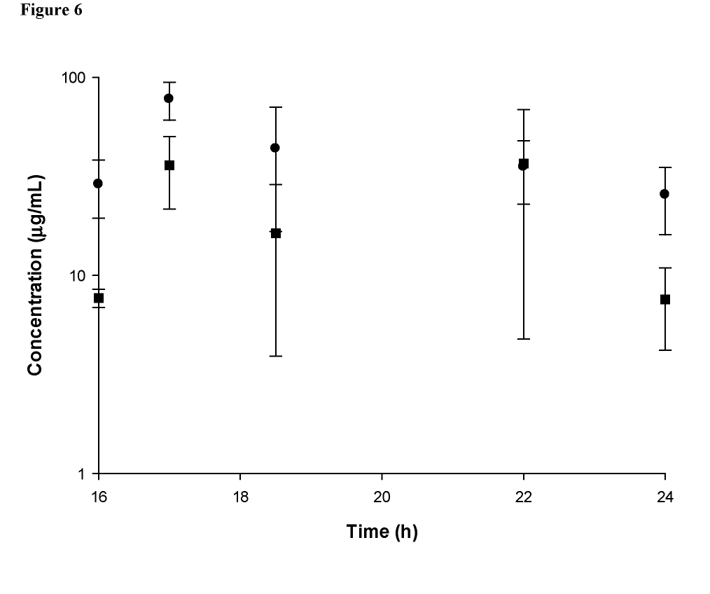

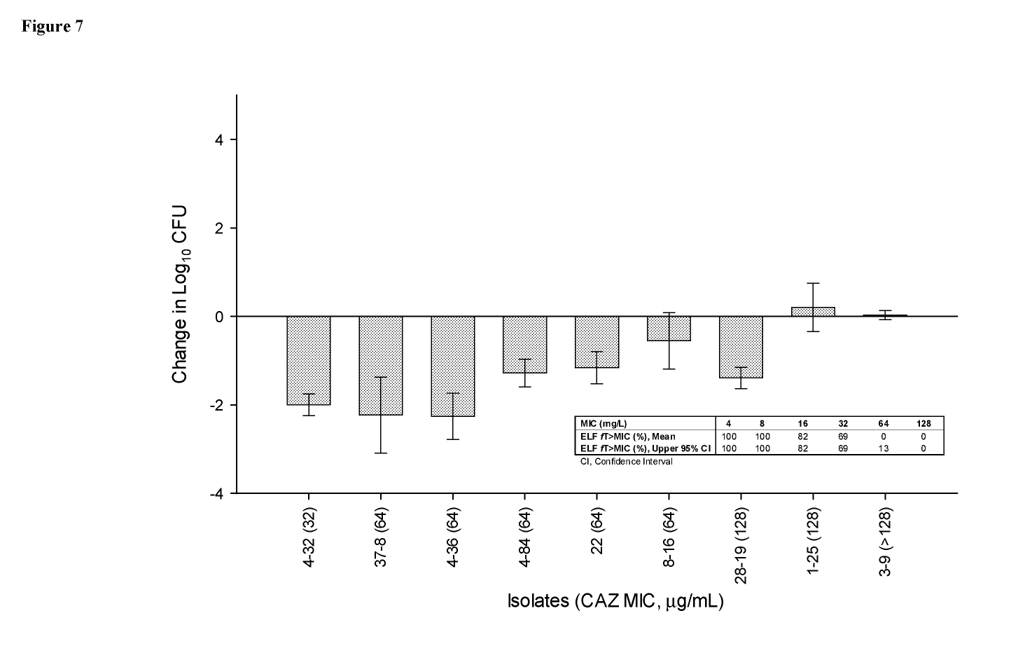

The free drug serum and ELF pharmacokinetic profiles determined in vivo for ceftazidime are shown in FIG. 6. During the conduct of the studies, it was determined this regimen would not produce sufficient range of fT>MIC within the ELF to observe the break in efficacy. As such, the efficacy data described and depicted in Table 9 and FIG. 7 was not used for any pharmacodynamic determinations.

TABLE 9
Results of the in vivo efficacy studies utilizing the ceftazidime
regimen in the neutropenic lung infection model. *0 h control data
representative of the bacterial density at the start of dosing.

| | | Time point | Change log CFU* | | # of |
|---|---|---|---|---|---|
| Isolate # | Group | (h) | Mean | SD | Lungs |
| PSA 22 | Control | 0 | 5.99* | 0.11 | 4 |
| | Control | 24 | 9.15 | 0.02 | 4 |
| | CAZ | 24 | 4.83 | 0.46 | 4 |
| PSA 37-8 | Control | 0 | 6.02* | 0.28 | 10 |
| | Control | 24 | 8.26 | 0.15 | 9 |
| | CAZ | 24 | 3.8 | 0.54 | 9 |
| PSA 4-32 | Control | 0 | 5.56* | 0.16 | 4 |
| | Control | 24 | 9.34 | 0.14 | 4 |
| | CAZ | 24 | 3.57 | 0.24 | 4 |
| PSA 8-16 | Control | 0 | 5.93* | 0.15 | 4 |
| | Control | 24 | 9.94 | 0.18 | 5 |
| | CAZ | 24 | 5.38 | 0.64 | 4 |
| PSA 28-19 | Control | 0 | 6.46* | 0.2 | 4 |
| | Control | 24 | 9.8 | 0.16 | 5 |
| | CAZ | 24 | 5.07 | 0.24 | 4 |
| PSA 4-36 | Control | 0 | 6.53* | 0.2 | 4 |
| | Control | 24 | 9.72 | 0.08 | 4 |
| | CAZ | 24 | 5.01 | 0.79 | 4 |
| PSA 4-84 | Control | 0 | 6.16* | 0.21 | 4 |
| | Control | 24 | 9.66 | 0.14 | 5 |
| | CAZ | 24 | 4.88 | 0.31 | 5 |
| PSA 1-25 | Control | 0 | 6.21* | 0.42 | 4 |
| | Control | 24 | 10.01 | 0.13 | 5 |
| | CAZ | 24 | 6.42 | 0.54 | 4 |
| PSA 3-9 | Control | 0 | 6.45* | 0.09 | 4 |
| | Control | 24 | 7.77 | 0.93 | 4 |
| | CAZ | 24 | 6.48 | 0.1 | 3 |

Determination of Dosing Regimen for Directed ELF fT>MIC Studies for Ceftazidime

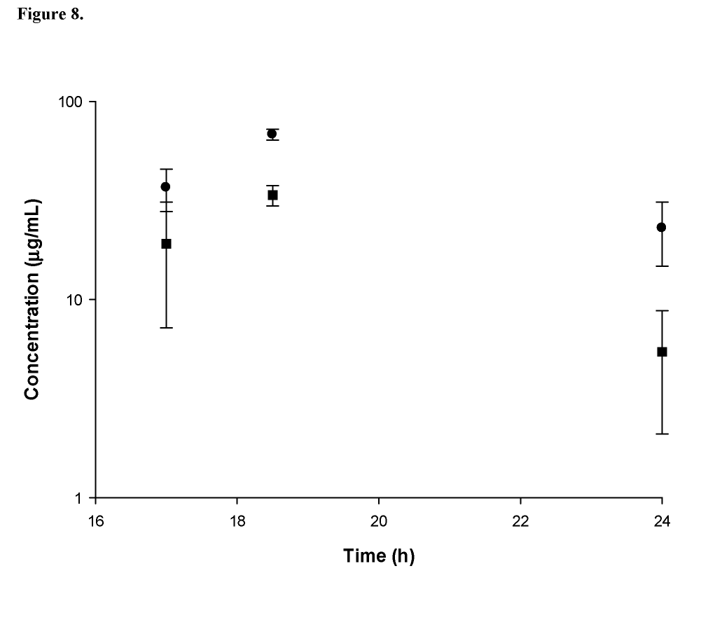

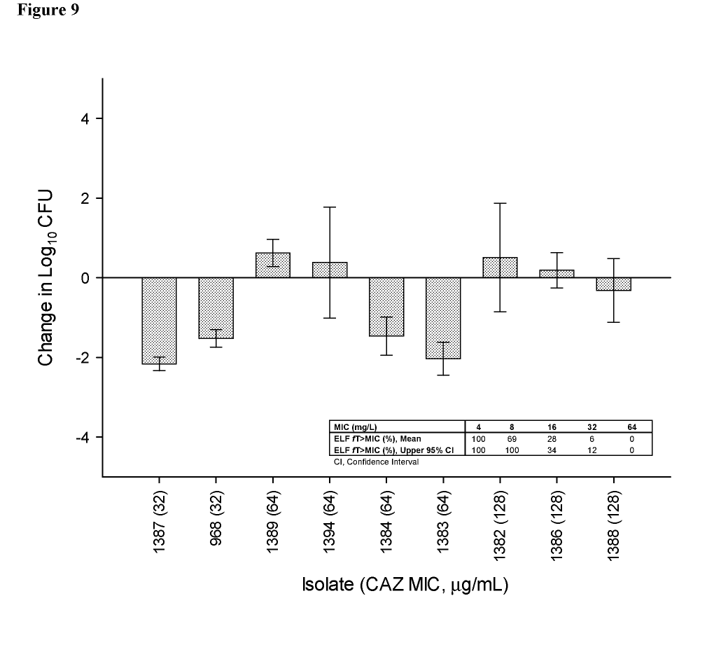

The free drug pharmacokinetic profile determined in vivo for ceftazidime alone is shown in FIG. 8. Further, the ELF concentrations over the third dosing interval for the above regimen are also shown in FIG. 8. From these data, fT>MIC values were determined against a range of MICs. The mean and upper 95% confidence interval are described in FIG. 9.

### In Vivo Efficacy: Ceftazidime-Avibactam Human Simulated Serum Studies

Studies were conducted in neutropenic animals. Respective bacterial densities in control mice at the initiation of dosing were 5.98±0.44 log₁₀CFU, increasing to 9.13±0.80 log₁₀CFU after 24 hours. Results of the neutropenic studies are shown in Table 10 and FIG. 5. Against the [symbol] collection of P. aeruginosa, activity was observed for ceftazidime-avibactam against isolates with MICs of 32 μg/mL and less, with the exception of one isolate at an MIC of 16 μg/mL. Activity was not observed against the lone isolate with an MIC of 64 μg/mL. After plating the direct homogenate onto drug containing plates, no growth was observed signifying no development of resistance present.

TABLE 10
Results of the in vivo efficacy studies utilizing the ceftazidime-avibactam
regimen in the neutropenic lung infection model.
*0 h control data representative of the bacterial density at the start
of dosing.

| | | Timepoint | Change log CFU* | | # of |
|---|---|---|---|---|---|
| Isolate # | Group | (h) | Mean | SD | Lungs |
| PSA 22 | Control | 0 | 5.99* | 0.11 | 4 |
| | Control | 24 | 9.15 | 0.02 | 4 |
| | CAZ- | 24 | 2.42 | 0.63 | 5 |
| | AVI | | | | |
| PSA 971 | Control | 0 | 6.27* | 0.09 | 4 |
| | Control | 24 | 9.13 | 0.31 | 5 |
| | CAZ- | 24 | 5.12 | 1.18 | 4 |
| | AVI | | | | |
| PSA 1383 | Control | 0 | 6.04* | 0.1 | 4 |
| | Control | 24 | 8.13 | 0.08 | 4 |
| | CAZ- | 24 | 2 | 0 | 5 |
| | AVI | | | | |
| PSA 1384 | Control | 0 | 5.9* | 0.11 | 4 |
| | Control | 24 | 8.15 | 0.04 | 4 |
| | CAZ- | 24 | 3.95 | 0.61 | 5 |
| | AVI | | | | |
| PSA 37-8 | Control | 0 | 6.02* | 0.28 | 10 |
| | Control | 24 | 8.26 | 0.15 | 9 |
| | CAZ- | 24 | 4.38 | 0.68 | 9 |
| | AVI | | | | |
| PSA 856 | Control | 0 | 6.11* | 0.44 | 5 |
| | Control | 24 | 9.9 | 0.17 | 5 |
| | CAZ- | 24 | 4.93 | 0.45 | 4 |
| | AVI | | | | |
| PSA 1382 | Control | 0 | 6.06* | 0.22 | 5 |
| | Control | 24 | 9.42 | 0.08 | 5 |
| | CAZ- | 24 | 4.7 | 0.71 | 4 |
| | AVI | | | | |
| PSA 1386 | Control | 0 | 5.91* | 0.18 | 3 |
| | Control | 24 | 9.93 | 0.11 | 5 |
| | CAZ- | 24 | 3.82 | 0.51 | 4 |
| | AVI | | | | |
| PSA 1387 | Control | 0 | 6.34* | 0.32 | 5 |
| | Control | 24 | 9.84 | 0.15 | 4 |
| | CAZ- | 24 | 3.89 | 0.39 | 4 |
| | AVI | | | | |
| PSA 1388 | Control | 0 | 6.13* | 0.14 | 4 |
| | Control | 24 | 9.3 | 0.25 | 5 |
| | CAZ- | 24 | 3.61 | 0.56 | 5 |
| | AVI | | | | |
| PSA 1389 | Control | 0 | 5.82* | 0.2 | 5 |
| | Control | 24 | 10.09 | 0.13 | 5 |
| | CAZ- | 24 | 3.75 | 0.27 | 3 |
| | AVI | | | | |
| PSA 4-32 | Control | 0 | 5.56* | 0.16 | 4 |
| | Control | 24 | 9.34 | 0.14 | 4 |
| | CAZ- | 24 | 3.31 | 0.2 | 5 |
| | AVI | | | | |
| PSA 4-39 | Control | 0 | 5.82* | 0.12 | 5 |
| | Control | 24 | 7.96 | 0.24 | 4 |
| | CAZ- | 24 | 3.78 | 0.24 | 4 |
| | AVI | | | | |
| PSA 2-69 | Control | 0 | 5.38* | 0.23 | 4 |
| | Control | 24 | 8.31 | 0.05 | 5 |
| | CAZ- | 24 | 3.61 | 0.35 | 4 |
| | AVI | | | | |
| PSA 8-16 | Control | 0 | 5.93* | 0.15 | 4 |
| | Control | 24 | 9.94 | 0.18 | 5 |
| | CAZ- | 24 | 3.8 | 0.47 | 3 |
| | AVI | | | | |
| PSA 24-2 | Control | 0 | 6.13* | 0.03 | 4 |
| | Control | 24 | 8.34 | 1.07 | 5 |
| | CAZ- | 24 | 2.84 | 0.37 | 5 |
| | AVI | | | | |
| PSA 28-19 | Control | 0 | 6.46* | 0.2 | 4 |
| | Control | 24 | 9.8 | 0.16 | 5 |
| | CAZ- | 24 | 4.6 | 0.35 | 4 |
| | AVI | | | | |
| PSA 968 | Control | 0 | 5.73* | 1.19 | 5 |
| | Control | 24 | 9.79 | 0.05 | 3 |
| | CAZ- | 24 | 4.79 | 0.47 | 4 |
| | AVI | | | | |
| PSA 1391 | Control | 0 | 5.86* | 0.09 | 5 |
| | Control | 24 | 9.28 | 0.06 | 3 |
| | CAZ- | 24 | 4.3 | 1.4 | 4 |
| | AVI | | | | |
| PSA 1394 | Control | 0 | 5.49* | 0.41 | 5 |
| | Control | 24 | 8.57 | 0.19 | 3 |
| | CAZ- | 24 | 3.55 | 0.44 | 4 |
| | AVI | | | | |
| PSA 4-36 | Control | 0 | 6.53* | 0.2 | 4 |
| | Control | 24 | 9.72 | 0.08 | 4 |
| | CAZ- | 24 | 3.56 | 0.86 | 5 |
| | AVI | | | | |
| PSA 4-84 | Control | 0 | 6.16* | 0.21 | 4 |
| | Control | 24 | 9.66 | 0.14 | 5 |
| | CAZ- | 24 | 4.72 | 0.38 | 5 |
| | AVI | | | | |
| PSA 1-25 | Control | 0 | 6.21* | 0.42 | 4 |
| | Control | 24 | 10.01 | 0.13 | 5 |
| | CAZ- | 24 | 5.51 | 0.94 | 5 |
| | AVI | | | | |
| PSA 1-29 | Control | 0 | 6.28* | 0.52 | 5 |
| | Control | 24 | 9.4 | 0.09 | 4 |
| | CAZ- | 24 | 4.89 | 0.85 | 4 |
| | AVI | | | | |
| PSA 3-9 | Control | 0 | 6.45* | 0.09 | 4 |
| | Control | 24 | 7.77 | 0.93 | 4 |
| | CAZ- | 24 | 7.26 | 0.91 | 4 |
| | AVI | | | | |
| PSA 11-54 | Control | 0 | 6.02* | 0.1 | 4 |
| | Control | 24 | 9.88 | 0.08 | 5 |
| | CAZ- | 24 | 4.46 | 0.43 | 3 |
| | AVI | | | | |
| PSA 7-6 | Control | 0 | 5.34* | 0.65 | 4 |
| | Control | 24 | 8.78 | 0.41 | 5 |
| | CAZ- | 24 | 3.19 | 0.26 | 4 |
| | AVI | | | | |
| PSA 14-28 | Control | 0 | 5.55* | 0.31 | 5 |
| | Control | 24 | 7.9 | 0.01 | 4 |
| | CAZ- | 24 | 6.52 | 0.31 | 5 |
| | AVI | | | | |

In Vivo Efficacy: Ceftazidime Directed ELF fT>MIC Studies

The results of the directed ELF fT>MIC efficacy studies are shown in Table 11 and FIG. 9. Studies were conducted in neutropenic animals. Respective bacterial densities in control mice at the initiation of dosing were 5.89±0.30 and log₁₀CFU, increasing to 8.75±0.93 log₁₀CFU after 24 hours. Isolates were chosen based on the ceftazidime MIC between the range of 32 and 128 μg/mL. Activity was observed against those isolates with MICs of 32 μg/mL. Efficacy was variable against isolates with MICs of 64 μg/mL; isolates with MICs of 128 μg/mL saw little to no activity when using the ceftazidime monotherapy regimen.

TABLE 11
Results of the in vivo efficacy studies utilizing the directed
ELF fT > MIC ceftazidime regimen in the neutropenic lung infection
model. *0 h control data representative of the bacterial density at the start
of dosing.

| | | | Change log CFU* | | # of |
|---|---|---|---|---|---|
| Isolate # | Group | Timepoint (h) | Mean | SD | Lungs |
| PSA 1384 | Control | 0 | 5.86* | 0.14 | 5 |
| | Control | 24 | 7.61 | 0.16 | 4 |
| | CAZ | 24 | 4.4 | 0.48 | 4 |
| PSA 1383 | Control | 0 | 5.77* | 0.09 | 4 |
| | Control | 24 | 7.29 | 0.48 | 4 |
| | CAZ | 24 | 3.73 | 0.42 | 5 |
| PSA 1386 | Control | 0 | 5.79* | 0.4 | 5 |
| | Control | 24 | 9.67 | 0.12 | 5 |
| | CAZ | 24 | 5.98 | 0.44 | 6 |
| PSA 1388 | Control | 0 | 5.86* | 0.05 | 4 |
| | Control | 24 | 8.39 | 0.17 | 5 |
| | CAZ | 24 | 5.54 | 0.8 | 3 |
| PSA 968 | Control | 0 | 5.98* | 0.29 | 5 |
| | Control | 24 | 9.76 | 0.07 | 5 |
| | CAZ | 24 | 4.45 | 0.22 | 5 |
| PSA 1387 | Control | 0 | 6.39* | 0.19 | 5 |
| | Control | 24 | 9.45 | 0.3 | 5 |
| | CAZ | 24 | 4.23 | 0.17 | 3 |
| PSA 1389 | Control | 0 | 5.68* | 0.21 | 4 |
| | Control | 24 | 9.67 | 0.06 | 5 |
| | CAZ | 24 | 6.3 | 0.34 | 4 |
| PSA 1382 | Control | 0 | 5.91* | 0.23 | 4 |
| | Control | 24 | 8.64 | 0.31 | 5 |
| | CAZ | 24 | 6.42 | 1.37 | 5 |
| PSA 1394 | Control | 0 | 5.58* | 0.09 | 3 |
| | Control | 24 | 7.73 | 0.28 | 5 |
| | CAZ | 24 | 5.96 | 1.4 | 5 |

### Conclusion

Within the murine lung infection model, the combination of ceftazidime-avibactam produces considerable concentrations within the lung irrespective of host immune status. Utilizing a previously determined regimen of ceftazidime-avibactam, efficacy was observed against those isolates with MICs≦32 μg/mL, where ELF fT>MIC≧19%. Given these observations, the needed fT>MIC within the lung, may be less than previously thought for cephalosporins, approximately 60%. Certainly where there is no noticeable ELF fT>MIC, at MICs of 64 μg/mL for the combination, ceftazidime-avibactam does not produce efficacy. The results of the ceftazidime [symbol] directed ELF fT>MIC also demonstrate this may be true with activity observed against MICs of 32 μg/mL, where ELF fT>MIC is 12% and variable to no activity at MICs of 64 and 128 μg/mL.

### Example 4

### Neutropenic Thigh Model

CD-1 neutropenic mice were infected with appr. 10⁶ cfu β-lactamase-producing P. aeruginosa strains in the thigh. Treatment was started after 2 h with ceftazidime alone (q2h with various doses) for 24 h and cfu determined in the thigh to establish its exposure response relationship. Full dose fractionation studies of avibactam were performed for 2 strains (ceftazidime MICs 32 and 64 mg/L). The exposure response of avibactam q2h was determined for another 6 P. aeruginosa strains (ceftazidime MICs 64-128 mg/L). The Eₘₐₓ model was fit to the dose and PK/PD index (PDI) responses to determine the PDI values of ceftazidime alone and in combination with avibactam resulting in a static effect, a 1- and 2-log₁₀ kill. For avibactam the % time of the dosing interval above a virtual in vivo MIC (threshold concentrations, C_{T}) % fT>C_{T} were calculated (C_{T} of 0.25, 1 and 4 mg/L).

The static % fT>MIC of ceftazidime for monotherapy was between 0 and 38%, with some strains requiring the lower % fT>MICs. Avibactam reduced the ceftazidime static % fT>MIC for all strains. In the dose fractionation studies, the best PDI correlation for avibactam was observed for % fT>C_{T} of 1 mg/L. At a C_{T} of 1 mg/L, a % fT>C_{T} of 30.2-74.1 was required. For another 6 strains the mean % fT>C_{T} 1 mg/L was 36.3% (14.1-62.5). The estimates required for a static effect were partly dependent on the dose of ceftazidime (a lower value required if the ceftazidime dose was higher). The effect of avibactam was primarily dependent on the time above threshold % fT>C_{T} 1 mg/L, with a mean value of 36.3% when given concomitantly with ceftazidime. Relatively higher exposures were required at lower doses of ceftazidime.

### Materials and Methods

### Antibacterials:

### Ceftazidime (CAZ)

### Provided by AstraZeneca (ex GSK)

### Lot no.: G263770,

Exp. Date: 5 Dec. 2012

### Date of Manufacture: 6 Dec. 2010

### CAS no: 78439-06-2

### Potency: 77.2%

### Avibactam

### AstraZeneca (ex Dr Reddy)

Lot no.: AFCH005151 (07113P028), Analytical number: A1002CQ055
Exp. Date: March 2013

### Potency: 91.7%

### Bacterial Strains and Susceptibility Testing:

Seven well-characterized ceftazidime-resistant Pseudomonas aeruginosa strains, obtained from a variety of clinically relevant sources, were used in the experiments as shown in Table 1. MICs were taken from an earlier in vitro checkerboard study (Berkhout & Mouton 2013 CAZ-AVI-M1-061) and were determined by microdilution according to the ISO-guidelines (ISO 2006). This method is CLSI compatible.

### Animals:

Outbred female CD-1 mice (Charles River, Netherlands), 4 to 5 weeks old, weighing 20 to 25 g were used in the experiments. Granulocytopenia was induced by two doses of cyclophosphamide s.c.: one at four days (150 mg/kg) and the other at one day (100 mg/kg) before the infection experiment.

The animals were housed under standard conditions with drink and feed supplied ad libitum and were examined once daily and after immunosuppression 2-3 times per day. The animal studies were conducted in accordance with the recommendations of the European Community (Directive 86/609/EEC, 24 Nov. 1986), and all animal procedures were approved by the Animal Welfare Committee of Radboud University (RU-DEC 2012-003).

### Infection:

The neutropenic mice were infected with 2 P. aeruginosa strains per animal, one in the left and one in the right thigh. 0.05 mL of bacterial suspension consisting of approximately 10⁵-10⁶ bacteria was inoculated intramuscularly. Treatment with increasing doses of ceftazidime or saline (control), every dose being 0.1 mL administered subcutaneously, was started at t=0 h, 2 hours after initiation of infection, with a dosing regimen of q2h that continued for a period of 24 h. All dosing regimens were performed in at least two animals. At t=0 h, 2 mice were humanely sacrificed to determine the initial inoculum just before treatment. All other animals were sacrificed at t=24 h unless the welfare of the animals indicated earlier termination was necessary, following animal welfare regulations. Thighs were taken and moved to a pre-cooled 10-mL plastic tube (Transport Tube, Omnilabo, NL) [symbol] containing 2 mL phosphate buffered saline (PBS; NaCl 8.00 g/L, Na₂HPO₄2H₂ 1.44 g/L, KH₂PO₄ 0.26 g/L, pH 7.2-7.4). Subsequently thighs were ground using an Ultra-Turrax (IKA Labortechnik, Germany). A tenfold dilution series was prepared and 3×10 μL plated (Chromagar, Biomerieux, NL) per dilution. The following day, colonies were counted and the number of cfu per thigh calculated. The drug effect was then determined as the difference between the log₁₀[cfu/thigh] values at t=24 h and t=0 h (mean value of 2 mice) expressed as “dcfu”.

### Antibiotic Concentration Measurements

Blood was separated from plasma using a cooled centrifuge. Samples were split and stored at −80° C. Concentrations of ceftazidime and avibactam were determined by the Drug Metabolism and Pharmacokinetics group in AstraZeneca (Waltham, Mass.) with a lower limit of quantitation of 1.5 ng/mL for ceftazidime and 1.8 ng/mL for avibactam. Protein binding was 10% for ceftazidime and 8% for avibactam as determined in the equilibrium dialysis chamber and analysed via LC-MS/MS.

### Evaluation and Validation of Combined Dosing Schemes

The exposure-response relationship of avibactam in neutropenic mice with experimental thigh infection was determined under treatment with a fixed dosing regimen of ceftazidime resulting in a 1 to 2 log₁₀ cfu increase as compared to the initial inoculum of the particular strain after 24 h of ceftazidime treatment. This regimen was chosen because of the sensitivity to changes in effects of avibactam. The amount of avibactam administered varied in frequency and dose. Exposures of ceftazidime and avibactam were determined using MicLab 2.36 (Medimatics, Maastricht, The Netherlands) using pharmacokinetic parameter estimates obtained from pharmacokinetic studies. In the simulations a protein binding was used of 10% for ceftazidime and 8% for avibactam. The drug effect was determined as the difference between the log₁₀ [cfu/thigh] values at t=24 h and t=0 h (mean value of 2 mice) expressed as dcfu. Free drug concentrations were used in all calculations. The Eₘₐₓ model (or linear regression) was fit to the dose and PK/PD index (PDI) responses to determine the PDI values of ceftazidime alone and in combination with avibactam resulting in a static, or particular specified log-kill, effect. For avibactam % fT>C_{T}, the % time of the dosing interval above threshold concentration C_{T} were calculated for C_{T} of 0.25, 1 and 4 mg/L. The C_{T} values were chosen based on the activity of avibactam in vitro, 4 mg/L being used in susceptibility testing, but lower concentrations also being active as determined in in vitro checkerboard studies and in a hollow fiber model with Enterobacteriaceae (Nichols W, Levasseur P, Li J, Das S. 2012. A threshold concentration of avibactam (AVI) during the pharmacokinetic decline phase, below which β-lactamase inhibition in Enterobacteriaceae becomes ineffective. Oral presentation at the [symbol] 52^{nd} Interscience Conference on Antimicrobial Agents and Chemotherapy, San Francisco, Calif. Abstract #A-1760).

TABLE 12
P. aeruginosa strains used for pharmacodynamic studies of ceftazidime and avibactam

| | | MIC CAZ | MIC CAZ-AVI* | |
|---|---|---|---|---|
| Isolate no. | resistance summary (if known) | mg/L | mg/L | static % fT > MIC (CAZ) |
| 1 | nitrocefinase activity ‘++’; AmpC transcript | 128 | 8 | no effect |
| | overexpressed; β-lactamase genotype: bla_{AmpC}, | | | |
| | bla_{PoxB}; class A-, class B- | | | |
| 3 | nitrocefinase activity baseline; AmpC transcript basal; | 64 | 2 | 0 |
| | β-lactamase genotype: bla_{AmpC}, bla_{PoxB}, bla_{TEM-24 (CAZ-6)}, | | | |
| | class B- | | | |
| 5 | nitrocefinase activity ‘++++’; AmpC transcript | 128 | 8 | 3 |
| | overexpressed; β-lactamase genotype: bla_{ampC}, | | | |
| | bla_{poxB}; class A-, class B- | | | |
| 7 | nitrocefinase activity ‘+++’; AmpC transcript | 64 | 4 | 7.2 |
| | overexpressed; β-lactamase genotype: bla_{ampC}, | | | |
| | bla_{poxB}; class A-, class B- | | | |
| 11 | OprD-, AmpCcon, Class A-, Class B- | 128 | 16 | no effect |
| 18 | OprD-, AmpCind?, Class A-, Class B- | 32 | 2 | 9 |
| 19 | OprD-, AmpCcon, Class A-, Class B- | 64 | 4 | 38 |

The Pseudomonas aeruginosa strains used in the experiments were selected based on MICs in vitro and the results of the checkerboards in vitro.
CAZ = ceftazidime,
CAZ-AVI = combination of ceftazidime and avibactam,
*MIC CAZ-AVI determined at 4 mg/L avibactam.

TABLE 13
Design of dose fractionation experiments.
Example of a design of dose fractionations for treatment of neutropenic CD-1 female mice with
combined regimens of ceftazidime and avibactam. Each group consisted of 2 mice and received a
fixed dose of ceftazidime every 2 hours based on a 2 log₁₀ increase compared to the initial
inoculum and avibactam as displayed in the schedule.

| | REAL TIME | | | | | | | | | | | |
|---|---|---|---|---|---|---|---|---|---|---|---|---|
| GROUP | 12 uur | 14 uur | 16 uur | 18 uur | 20 uur | 22 uur | 24 uur | 2 uur | 4 uur | 6 uur | 8 uur | 10 uur |
| Gr 1 | 64 | 0 | 0 | 0 | 0 | 0 | 0 | 0 | 0 | 0 | 0 | 0 |
| Gr 2 | 64 | 0 | 0 | 0 | 0 | 0 | 64 | 0 | 0 | 0 | 0 | 0 |
| Gr 3 | 64 | 64 | 64 | 64 | 64 | 64 | 64 | 64 | 64 | 64 | 64 | 64 |
| Gr 4 | 32 | 0 | 0 | 0 | 0 | 0 | 0 | 0 | 0 | 0 | 0 | 0 |
| Gr 5 | 32 | 0 | 0 | 0 | 0 | 0 | 32 | 0 | 0 | 0 | 0 | 0 |
| Gr 6 | 32 | 0 | 0 | 32 | 0 | 0 | 32 | 0 | 0 | 32 | 0 | 0 |
| Gr 7 | 16 | 0 | 0 | 0 | 0 | 0 | 0 | 0 | 0 | 0 | 0 | 0 |
| Gr 8 | 16 | 0 | 0 | 0 | 0 | 0 | 16 | 0 | 0 | 0 | 0 | 0 |
| Gr 9 | 16 | 0 | 0 | 16 | 0 | 0 | 16 | 0 | 0 | 16 | 0 | 0 |
| Gr 10 | 16 | 0 | 16 | 0 | 16 | 0 | 16 | 0 | 16 | 0 | 16 | 0 |
| Gr 11 | 8 | 0 | 0 | 0 | 0 | 0 | 0 | 0 | 0 | 0 | 0 | 0 |
| Gr 12 | 8 | 0 | 0 | 0 | 0 | 0 | 8 | 0 | 0 | 0 | 0 | 0 |
| Gr 13 | 8 | 0 | 0 | 8 | 0 | 0 | 8 | 0 | 0 | 8 | 0 | 0 |
| Gr 14 | 8 | 8 | 0 | 8 | 8 | 0 | 8 | 8 | 0 | 8 | 8 | 0 |
| Gr 15 | 4 | 0 | 0 | 0 | 0 | 0 | 0 | 0 | 0 | 0 | 0 | 0 |
| Gr 16 | 4 | 0 | 0 | 4 | 0 | 0 | 4 | 0 | 0 | 4 | 0 | 0 |
| Gr 17 | 4 | 0 | 4 | 0 | 4 | 0 | 4 | 0 | 4 | 0 | 4 | 0 |
| Gr 18 | 4 | 4 | 0 | 4 | 4 | 0 | 4 | 4 | 0 | 4 | 4 | 0 |
| Gr 19 | 4 | 4 | 4 | 4 | 4 | 4 | 4 | 4 | 4 | 4 | 4 | 4 |
| Gr 20 | C2 hrs | — | — | — | — | — | — | — | — | — | — | — |
| Gr 21 | 0 | 0 | 0 | 0 | 0 | 0 | 0 | 0 | 0 | 0 | 0 | 0 |
| Gr 22 | FZ | FZ | FZ | FZ | FZ | FZ | FZ | FZ | FZ | FZ | FZ | FZ |

Gr, Group;
FZ, physiological saline;
uur, hour;
C, control.
Doses in mg/kg

TABLE 14
Exposures of avibactam associated with a 24-h response of stasis for 6 strains of
P. aeruginosa in the treatment of thigh infected mice when combined with a pre-
determined constant dosing of ceftazidime.

| Strain | MIC mg/L | Dose CAZ mg/kg | TDD AVI mg/kg | log TDD AVI | % fT > C_{T}1 |
|---|---|---|---|---|---|
| 7 | 64 | 64 | 267.47 | 2.43 | 50.4 |
| 18 | 32 | 64 | 58.91 | 1.77 | 24.2 |
| 5 | 128 | 64 | 33.59 | 1.53 | 14.1 |
| 11 | 128 | 64 | 77.88 | 1.89 | 29.1 |
| 1 | 128 | 32 | 123.70 | 2.09 | 37.2 |
| 19 | 64 | 32 | 541.34 | 2.73 | 62.5 |

| | |
|---|---|
| mean | 36.3 |
| sd | 17.8 |

C_{T}1 = threshold concentration of 1 mg/L,
TDD = total daily dose,
CAZ = ceftazidime,
AVI = avibactam

TABLE 15
Non-linear regression (Eₘₐₓ model) results of treatment of mice with ceftazidime
q2h and avibactam q2h

| Strain | 1 | | 5 | 7 | 11 | 18 | 19 |
|---|---|---|---|---|---|---|---|
| maximum | 1.26 | (1.44) | ≈0.70 | 1.81 (0.29) | 1.40 (0.50) | 0.83 (0.36) | 2.46 (0.22) |
| minimum | ≈′−727.5 | (≈605114) | −3.25 (0.95) | −2.83 (1.41) | −2.95 (0.41) | −2.36 (0.23) | −1.42 (0.46) |

| | | | | | | |
|---|---|---|---|---|---|---|
| EC50 | ≈8.14e9 | 123.00 | 417.90 | 113.50 | 88.34 | 491.00 |
| Hill slope | ≈′−0.35) | −1.185 (0.86) | −1.01 (0.41) | −1.98 (0.86) | −2.60 (1.16) | −5.75 (3.57) |

SE between brackets

### Results

As displayed in Table 12, the static % fT>MIC of ceftazidime during monotherapy was between 0 and 38%. For the most resistant strains, no effect could be observed, requiring the lower % fT>MICs. This indicates that less % fT>MIC is necessary for some highly resistant strains. Avibactam reduced the static % fT>MIC of ceftazidime for all strains.

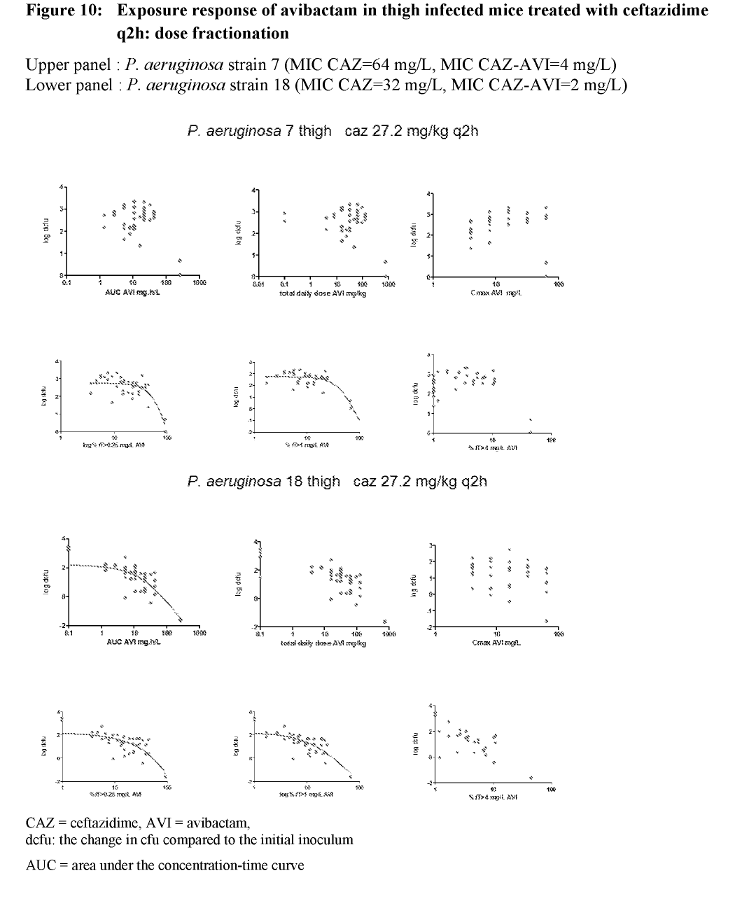

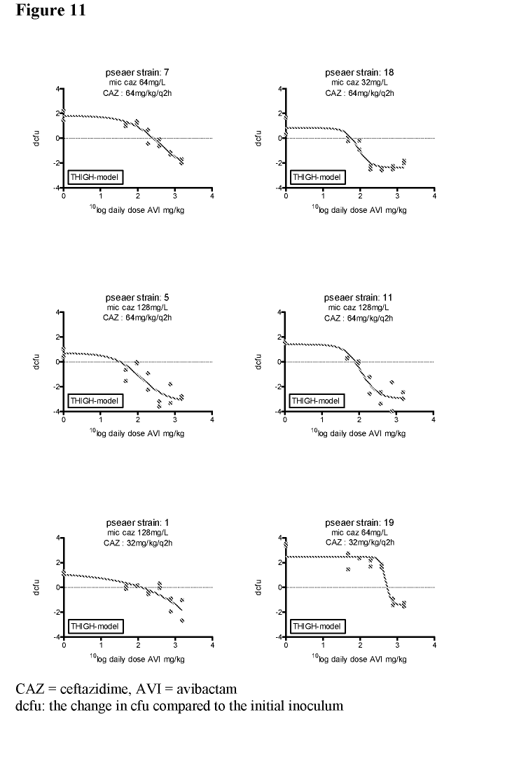

The design of the full dose fractionation studies is shown in Table 13. FIG. 10 shows the results of two strains submitted to a full dose fractionation study of avibactam. Although each of the indices Cₘₐₓ, AUC/MIC and dose showed some correlation, visual inspection of the figures leads to the conclusion % fT>C_{T} is a somewhat better predictor, but there appears to be significant variation. This indicates that % fT>C_{T} is not the only factor that determines outcome in this setting. The figure shows the % fT>C_{T} for three concentrations of the C_{T}. The % fT>C_{T} 1 mg/L appears to be a somewhat better predictor than 0.25 mg/L and 4 mg/L. At a concentration of 1 mg/L, a % fT>C_{T} of 30.2 (strain 7) or 74.1 (strain 18) was required to support a bacteriostatic effect. The doses required to reach a static effect with varying doses of avibactam with a fixed dosing regimen of ceftazidime were determined for 6 strains (FIG. 11). From these results, % fT>C_{T} 1 [symbol] mg/L was determined for each strain. The mean % fT>C_{T} was 36.3% (14.1-62.5) (Table 14). The estimates required for a static effect were partly dependent on the dose of ceftazidime (a lower value was required if the ceftazidime dose was relatively higher with respect to the MIC of the strain).

Table 15 shows the results of the non-linear regression analysis for the 6 P. aeruginosa strains. The estimates show a significant standard error in some cases.

### Example 5

### Pharmacodynamics of Ceftazidime and Avibactam in Neutropenic Female CD-1 Mice with Experimental Pneumonia

CD-1 neutropenic mice were infected with approx. 10⁶ cfu in the lung by instillation through the nares under light anaesthesia. Treatment was started after 2 h with ceftazidime alone (q2h with various doses) for 24 h and cfu determined in the lung to establish its exposure response relationship. Avibactam was given q2h or q8h for two strains with MICs for ceftazidime of 32 and 128 mg/L respectively in twofold increasing doses at ceftazidime exposures that in ceftazidime monotherapy experiments had been the maximum exposures that had allowed 2 log₁₀ growth. Full dose fractionation studies of avibactam were performed for 2 strains at two different dose levels of ceftazidime; in addition the efficacy of avibactam q2h was determined for another 2 strains. The Eₘₐₓ model was fit to the dose and PK/PD index (PDI) responses to determine the PDI values of ceftazidime alone and in combination with avibactam resulting in a static effect, a 1- and a 2-log kill. For avibactam the % time of the dosing interval above a virtual in vivo threshold concentration, C_{T}, % fT>C_{T}, were calculated (C_{T} of 0.25, 1 and 4 mg/L).

Exposure response relationships for avibactam (R² 0.54-0.86) indicated that q2h was more efficacious than q8h, reducing the daily dose by factors of 2.7 and 10.1 for the 2 strains to obtain a static effect of the combination. This corresponds to a mean % fT>C_{T} 1 mg/L of 20.1 (range 16.1-23.5). In the dose fractionation study, the best PDI correlation for avibactam was observed for % fT>C_{T} 1 mg/L. The avibactam exposure estimates required for a static effect were partly dependent on the dose of ceftazidime (a lower value required if the ceftazidime dose was higher). For two control strains the % fT>C_{T} 1 mg/L estimates were 22.4 and 21.6%.

In conclusion, the effect of avibactam was dependent on the dose frequency; a decreased effect was observed with decreased frequency. The main PK/PD index correlated to effect was time above threshold C_{T}. For most strains, the % fT>C_{T} of 1 mg/L for a static effect was between 16 and 25%. To define the minimum effect concentration of avibactam a new pharmacadynamic index is introduced based on a threshold concentration C_{T}. This parameter represents the threshold concentration of avibactam to result in significant effect in vivo. Consequently, the exposure of avibactam that is required for pharmacodynamic effects can be expressed using this parameter.

[symbol]

Thus, the exposure of avibactam is expressed as the pharmacodynamic index % fT>C_{T}, analogous to % fT>MIC of the β-lactam, in this study ceftazidime. Similar to ceftazidime, the estimate of the % fT>C_{T} depends on the C_{T} itself. But whereas the MIC of the β-lactam is usually known from in vitro data, the C_{T} is not. In the experiments presented here, test values of C_{T} were used: 0.25, 1 and 4 mg/L, in order empirically to select an optimal value. A theoretical value is currently not known.

In this study, the neutropenic mouse model with a lung infection was used to determine the exposure-response relationship of avibactam with a fixed dosing frequency of ceftazidime every 2 h for P. aeruginosa bij comparing different dosing regimens of avibactam.

### Materials and Methods

### Antibacterials:

### Ceftazidime (CAZ)

### Provided by AstraZeneca (ex GSK)

### Lot no.: G263770,

Exp. Date: 5 Dec. 2012

### Date of Manufacture: 6 Dec. 2010

### CAS no: 78439-06-2

### Potency: 77.2%

### Avibactam

### AstraZeneca (ex Dr Reddy)

Lot no.: AFCH005151 (07113P028), Analytical number: A1002CQ055
Exp. Date: March 2013

### Potency: 91.7%

### Bacterial Strains and Susceptibility Testing:

Seven well-characterized ceftazidime-resistant Pseudomonas aeruginosa strains, obtained from a variety of clinically relevant sources, were used in the experiments as shown in Error! Reference source not found. MICs were determined in an earlier in vitro checkerboard study (AstraZeneca study report # CAZ-AVI-M1-061) and were determined by microdilution according to the ISO-guidelines (ISO 2006). This method is CLSI compatible.

### Animals:

Outbred female CD-1 mice (Charles River, Netherlands), 4 to 5 weeks old, weighing 20 to 25 g were used in the experiments. Granulocytopenia was induced by two doses of cyclophosphamide s.c. four days (150 mg/kg) and one day (100 mg/kg) before the infection experiment.

The animals were housed under standard conditions with drink and teed supplied ad libitum and were examined once daily and after immunosuppression 2-3 times per day. The animal studies were conducted in accordance with the recommendations of the European Community (Directive 86/609/EEC, 24 Nov. 1986), and all animal procedures were approved by the Animal Welfare Committee of Radboud University (RU-DEC 2012-003).

### Infection:

Neutropenic mice were infected with one P. aeruginosa strain per animal. 0.05 mL of bacterial suspension consisting of approximately 10⁵-10⁶ bacteria was inoculated intra-nasally with a syringe under light anaesthesia with isoflurane. Treatment with increasing doses of 0.1 mL ceftazidime or saline (control) administered subcutaneously was started at 2 hours after initiation of infection (t=0 h) with a dosing regimen of q2h that continued for a period of 24 h. All dosing regimens were performed in at least two animals. At t=0 h, 2 mice were humanely sacrificed to determine the initial inoculum just before starting treatment. All other animals were sacrificed at t=24 h unless the welfare of the animals indicated earlier termination was necessary, following animal welfare regulations. Lungs were taken and moved to a pre-cooled 10-mL plastic tube (Transport Tube, Omnilabo, NL) containing 2 mL phosphate buffered saline (PBS; NaCl 8.00 g/L, Na₂HPO₄*2 H₂O 1.44 g/L, KH₂PO₄ 0.26 g/L, pH 7.2-7.4). Subsequently lungs were grounded using an Ultra-Turrax (IKA Labortechnik, Germany). A tenfold dilution series was prepared and 3×10 μL plated (Chromagar, Biomerieux, NL) per dilution. The following day, colonies were counted and the number of cfu per lung calculated.

### Data Analysis:

The exposure-response relationship of avibactam in neutropenic mice with experimental pneumonia was determined under treatment with a fixed dosing regimen of the highest dose of ceftazidime that resulted in a 1- to 2-log₁₀ cfu increase as compared to the initial inoculum of the particular strain after 24 h of ceftazidime treatment. This regimen was chosen because of the sensitivity to changes in effects of avibactam. The amount of avibactam administered varied in frequency and dose. Exposures of ceftazidime and avibactam were determined using MicLab 2.36 software (Medimatics, Maastricht, The Netherlands) using pharmacokinetic parameter estimates obtained from pharmacokinetic studies (AstraZeneca study report # CAZ-AVI-M1-065). In the simulations a protein binding was used of 10% for ceftazidime and 8% for avibactam. The drug [symbol] effect was determined as the difference between the log₁₀ cfu values at t=24 h and t=0 h (mean value of 2 mice) expressed as dcfu. Free drug concentrations were used in all calculations. The Eₘₐₓ model (or linear regression) was fit to the dose and PK/PD index (PDI) responses to determine the PDI values of ceftazidime alone and, separately, of avibactam in combination with ceftazidime. For avibactam % fT>C_{T}, the % time of the dosing interval above threshold concentration C_{T} were calculated for C_{T} of 0.25, 1 and 4 mg/L. The C_{T} values were chosen based on the activity of avibactam in vitro, 4 mg/L being used in susceptibility testing, but lower concentrations also being active as determined in in vitro checkerboard studies.

### Results

Table 11 shows the characteristics of the strains used and the efficacy of monotherapy. The static % fT>MIC of ceftazidime during monotherapy was between 0 and 38%. For the most resistant strains, no effect could be observed. On the other hand less % fT>MIC appeared to be necessary for some highly resistant strains. Avibactam reduced the static % fT>MIC of ceftazidime for all strains.

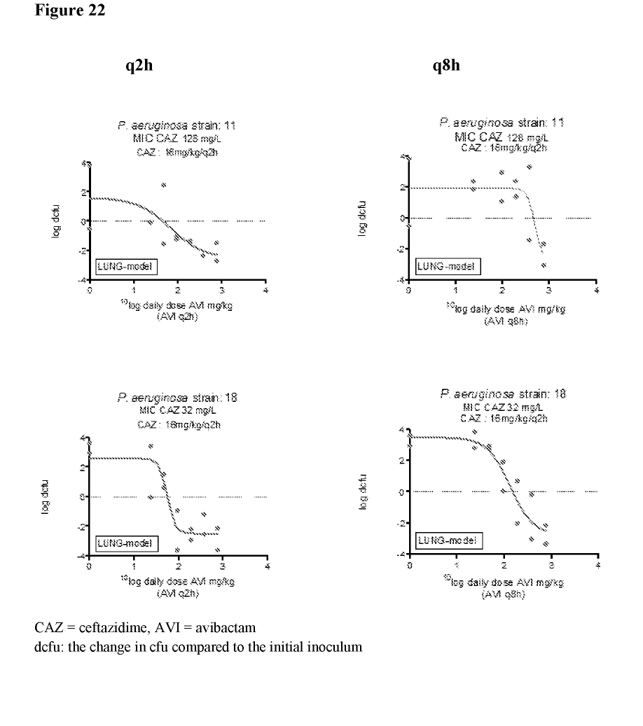

Exposure response curves (R² 0.54-0.86) for avibactam q2h or q8h indicated that the q2h regimen was more efficacious than the q8h regimen (FIG. 12). The daily dose of avibactam resulting in a static effect when mice were exposed to ceftazidime was lower for avibactam q2h than q8h by factors of 2.7 and 10.1 for strains 11 and 18, respectively. This corresponded to an overall mean % fT>C_{T} 1 mg/L for avibactam of 20.1% (range 16.1-23.5)(Table 16). Table 17 shows the parameter estimates of the Eₘₐₓ model fits.

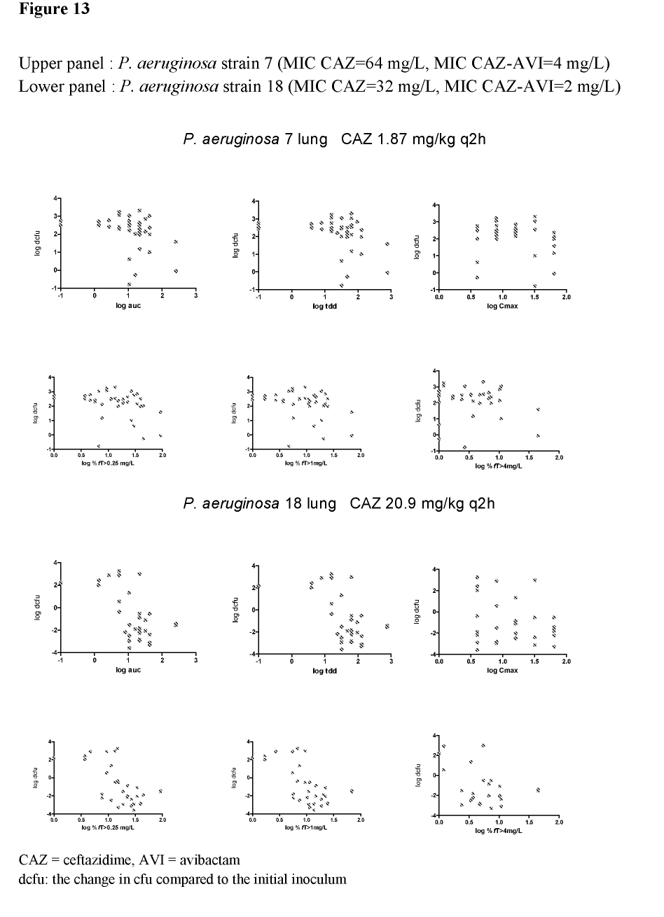

FIG. 13 shows the results of two strains submitted to a full dose fractionation study of avibactam. Although each of the indices Cₘₐₓ, AUC/MIC and dose showed some correlation, visual inspection of the figures led to the conclusion % fT>C_{T} was a somewhat better predictor, but there was significant variation. This indicates that % fT>C_{T} is not the only factor that determines outcome in this setting. The figure shows the % fT>C_{T} for three concentrations of the C_{T}. The % fT>C_{T} 1 mg/L appeared to be a somewhat better predictor than 0.25 mg/L and 4 mg/L. However, the exact threshold value cannot be determined from this figure. The estimates required for a static effect were partly dependent on the dose of ceftazidime (a lower value required if the ceftazidime dose was higher).

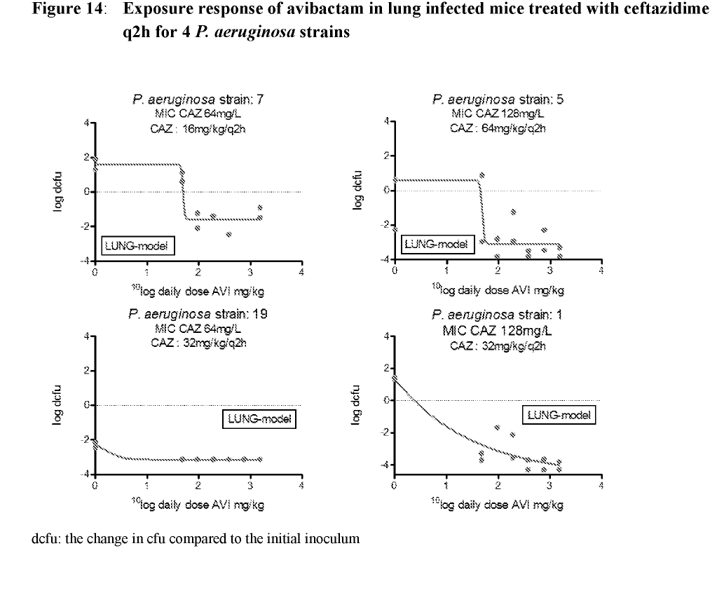

FIG. 14 shows the exposure response relationship of avibactam for four P. aeruginosa strains (7, 5, 19 and 1) when treated with various doses of ceftazidime q2h. Two of four P. aeruginosa strains showed more response to avibactam than expected as observed from the lower 2 panels. Table 18 provides the % fT>C_{T} 1 mg/L avibactam estimates of the four strains for the change in log₁₀(cfu/tissue sample)=0, or stasis over 24 h. For strains 1 and 19 that showed a better-than-predicted [symbol] effect, this could not be reliably estimated. For the other two strains it was 21.4 and 19.4%.

TABLE 16
% fT > C_{T} 1 mg/L of avibactam in mice treated with ceftazidime q2h and
avibactam q2h and q8h.

| | | Dose | TDD AVI | log TDD AVI | TDD AVI | log TDD AVI | | |
|---|---|---|---|---|---|---|---|---|
| | MIC | CAZ | q2h | q2h | q8h | q8h | % fT > C_{T}1 | % fT > C_{T}1 |
| Strain | mg/L | mg/kg | mg/kg | mg/kg | mg/kg | mg/kg | q2 | q8 |
| 18 | 32 | 16 | 56.65 | 1.75 | 150.57 | 2.18 | 23.5 | 16.1 |
| 11 | 128 | 16 | 45.65 | 1.66 | 463.29 | 2.67 | 19.7 | 20.9 |
| mean | | | | | | | 21.6 | 18.5 |
| sd | | | | | | | 2.7 | 3.4 |

C_{T} = threshold concentration (virtual in vivo inhibitory concentration),
TDD = total daily dose,
CAZ = ceftazidime,
AVI = avibactam

TABLE 17
Non-linear regression (Emax model) parameter estimates (SE) of effects
with treatment with ceftazidime q2h and avibactam q2h or q8h.

| | | |
|---|---|---|
| Strain | 18 | 11 |

q2h

| | | |
|---|---|---|
| maximum | 2.56 (0.68) | 1.60 (1.15) |
| minimum | −2.51 (0.53) | −2.57 (1.92) |
| EC₅₀ | 56.38 | 69.55 |
| Hill slope | −4.62 (4.11) | −1.13 (1.55) |

q8h

| | | |
|---|---|---|
| maximum | 3.45 (0.66) | 1.91 (0.73) |
| minimum | −2.79 (1.11) | −2.93 (23.38) |
| EC₅₀ | 133.30 | 505.80 |
| Hill slope | −1.77 (0.95) | −4.92 (52.91) |

1. A method of treating a nosocomial pneumonia infection in a patient in need thereof comprising administering to the patient an effective amount of the combination of ceftazidime or a pharmaceutically acceptable salt thereof, and avibactam, or a pharmaceutically acceptable salt thereof.

2. The method of claim 1, wherein the nosocomial pneumonia infection is caused by one or more pathogen expresses one or more beta-lactamase.

3. The method of claim 1, wherein the nosocomial pneumonia infection is not susceptible to ceftazidime as a mono-therapy.

4. The method of claim 1, wherein the nosocomial pneumonia infection is hospital acquired pneumonia.

5. The method of claim 1, wherein the nosocomial pneumonia infection is ventilator acquired pneumonia.

6. The method of claim 1, further comprising administering one or more additional therapeutic agent.

7. The method of claim 6, wherein the one or more additional therapeutic agent is selected from the group consisting of antibacterial agents, beta-lactamase inhibitors and antifungal agents.

8. The method of claim 7, wherein the one or more additional therapeutic agent is an antibacterial agent selected from the group consisting of tobramycin, levofloxacin, vancomyicn, linezolid, tigecycline and colistin.

9. The method of claim 1, wherein the combination of ceftazidime and avibactam are administered simultaneously.

10. The method of claim 1, wherein the combination of ceftazidime and avibactam are formulated independently and co-administered.

11. The method of any one of claim 1, wherein the combination of ceftazidime and avibactam are formulated independently and administered sequentially.

12. The method of claim 1, wherein the effective amount of the combination comprises about 2000 mg of ceftazidime and about 500 mg of avibactam per dose.

13. The method of claim 1, wherein the effective amount of the combination is administered approximately every eight hours.

14. The method of claim 1, wherein the effective amount of the combination is administered approximately every twelve hours.

15. The method of either one of claim 13, wherein the effective amount of the combination is administered intravenously.

16. The method of claim 15, wherein the effective amount of the combination is administered intravenously over the course of approximately 1 to 2 hours.

17. The method of claim 16, wherein the effective amount of the combination is administered intravenously over the course of approximately 2 hours.

18. The method of claim 16, wherein the effective amount of the combination is administered intravenously over the course of approximately 1 hour.

19. The combination of ceftazidime, or a pharmaceutically acceptable salt thereof, and avibactam, or a pharmaceutically acceptable salt thereof, for use as a medicament.

20. The combination of ceftazidime, or a pharmaceutically acceptable salt thereof, and avibactam, or a pharmaceutically acceptable salt thereof, for use in the treatment of a nosocomial pneumonia infection.

21. The combination of claim 20, wherein the nosocomial pneumonia infection is caused by one or more pathogens which express one or more beta-lactamase.

22. The combination of claim 20, wherein the nosocomial pneumonia infection is not susceptible to ceftazidime as a mono-therapy.

23. The combination of claim 20, wherein the nosocomial pneumonia infection is hospital acquired pneumonia.

24. The combination of claim 20, wherein the nosocomial pneumonia infection is ventilator acquired pneumonia.

25. The combination of claim 20, further comprising one or more additional therapeutic agent.

26. The combination of claim 25, wherein the additional therapeutic agent is selected from the group consisting of antibacterial agents, beta-lactamase inhibitors and antifungal agents.

27. The combination of claim 26, wherein the one or more additional therapeutic agent is an antibacterial agent selected from the group consisting of tobramycin, levofloxacin, vancomyicn, linezolid, tigecycline and colistin.

28. The combination of claim 20, wherein the combination of ceftazidime, or a pharmaceutically acceptable salt thereof, and avibactam, or a pharmaceutically acceptable salt thereof, are administered simultaneously.

29. The combination of claim 20, wherein the combination of ceftazidime, or a pharmaceutically acceptable salt thereof, and avibactam, or a pharmaceutically acceptable salt thereof, are independently formulated and co-administered.

30. The combination of claim 20, wherein the combination of ceftazidime, or a pharmaceutically acceptable salt thereof, and avibactam, or a pharmaceutically acceptable salt thereof, are independently formulated and administered sequentially.

31. The combination of claim 20, wherein the combination comprises about 2000 mg of ceftazidime, or a pharmaceutically acceptable salt thereof, and about 500 mg of avibactam, or pharmaceutically acceptable salt thereof, per dose.

32. The combination of claim 20, wherein the combination is administered approximately every eight hours.

33. The combination of claim 20, wherein the combination is administered approximately every twelve hours.

34. The combination of claim 20, wherein the combination is administered intravenously.

35. The combination of claim 34, wherein the combination is administered intravenously over the course of approximately 1 to 2 hours.

36. The combination of claim 35, wherein the combination is administered intravenously over the course of approximately 1 hour.

37. The combination of claim 35, wherein the combination is administered intravenously over the course of approximately 2 hours.
